(19) United States
(12) Patent Application Publication
(10) Pub. No.: US 2005/0032219 A1
(43) Pub. Date: Feb. 10, 2005

(54) Methods of administering vectors to synaptically connected neurons

(76) Inventors: Patrick Aubourg, Billancourt (FR); Nathalie Cartier-Lacave, Paris (FR); Elise Flavigny, Villeneuve Les Avignons (FR)

Correspondence Address: FINNEGAN, HENDERSON, FARABOW, GARRETT & DUNNER;LLP, 1300 I STREET, NW, WASHINGTON, DC, 20005, US

(21) Appl. No.: 10/482,696

(22) PCT Filed: Jul. 3, 2002
(86) PCT No.: PCT/IB02/03333

(30) Foreign Application Priority Data
Jul. 3, 2001 (US) 60302820

(51) Int. Cl. A61K 48/00; C12N 15/861

(52) U.S. Cl. 435/456; 424/93.2

(57) ABSTRACT

The present invention relates generally to efficient delivery of viral vectors to cells of the CNS, particularly useful in the treatment of neurodegenerative disorders and motor neuron diseases. The invention involves selecting a first population and a second population of synaptically connected neurons, wherein a therapeutic polypeptide is to be expressed in said second population of neurons; and administering rAAV virions comprising a therapeutic gene to said first subpopulation of neurons of said subject such that the rAAV virions are transported across a synapse between synaptically connected neurons. In another aspect the present invention also comprises the use of rAAV virions carrying a transgene in the preparation of a medicament for the treatment of a disease in a subject, wherein a first population and a second population of synaptically connected neurons are selected and a therapeutic polypeptide is to be expressed in said second population of neurons; and a medicament comprising recombinant adeno-associated virus (rAAV) virions is delivered to said first population of neurons of the subject, wherein said virions comprise a nucleic acid sequence that is expressible in transduced cells to provide a therapeutic effect in the subject, and wherein said rAAV virions are capable of transducing a synaptically connected neurons.

The present invention relates generally to efficient delivery of viral vectors to cells of the CNS. More particularly, the present invention relates to gene therapy for the treatment of central nervous system (CNS) disorders, particularly neurodegenerative disorders and motor neuron diseases.

Gene therapy to the central nervous system (CNS) involves the transfer and expression of therapeutic genes to prevent or slow down the degeneration of neurons or glial cells. Methods for gene delivery into the brain are either ex vivo, in which therapeutic genes are delivered in vitro to cells (encapsulated fibroblasts or myoblasts, neural stem cells) for subsequent transplantation into target brain regions, or in vivo, in which the therapeutic genes are directly transferred into the brain through a viral or non-viral vector.

Direct transfer of therapeutic genes into the brain faces significant hurdles because:
systemic in vivo delivery of gene therapy vectors results in limited transduction and transgene expression, even when one transiently disrupts the blood-brain barrier with hyperosmotic mannitol;
intraventricular injection of gene therapy vectors results only in transduction of ependymal and neuronal or glial cells that are close to the ventricles; and
direct stereotactic injection of gene therapy vectors results generally in transduction of neurons that are close to the injection site.

The only exception is the direct delivery of genes that code for soluble proteins (like lysosomal enzymes) that can be secreted by transduced cells at the site of injection and recaptured at distance through the endocytic pathway. The feasibility of this approach has been demonstrated in a mouse model of lysosomal enzyme deficiency (MPS VII) but it remains to be demonstrated to which extent the diffusion of soluble protein really occurs in the brain of larger animals (dog and monkey). In some CNS diseases, it may in addition be necessary to restrict the expression of therapeutic genes to specific neuronal or glial cell populations.

Parkinson's and Huntington's diseases are usually considered as paradigms for CNS gene therapy because they involve degeneration of specific and restricted brain regions. Thus, direct stereotactic injection of therapeutic genes in these specific brain regions is expected to allow sufficient expression of the therapeutic gene product, even with actual limitations gene therapy vectors that are currently used.

Given that the human brain contains approximately 10¹² neurons, CNS gene therapy seems at first view unfeasible in diseases like Alzheimer's and motor neurons diseases in which a diffuse gene delivery is required. However, these diseases are characterized by the degeneration of specific neuronal populations that are synaptically connected.

As long as integrity of axons connecting the different populations of neurons is conserved, preferably at an early stage of the disease, it should be possible to envisage a gene therapy approach that would aim to transfer therapeutic genes to one or few specific brain regions that contain neurons projecting to other specific brain or spinal cord neurons that are prone to degenerate. A crucial prerequisite of this approach is the existence of a therapeutic gene vector that can be transported from neurons to neurons through synapses. This is particularly important given that in most cases the therapeutic gene product will be a non-secreted protein. Even if the therapeutic gene product can be secreted (at the neuronal surface or at the synapse), such gene transfer would ensure a more diffuse delivery. In addition, such an approach would provide more specific delivery of the therapeutic gene.

Several vector systems and/or therapy regimes have been developed to address these issues, but significant disadvantages remain. One in vivo approach was based on the use of the neurotropic Herpes Simplex Virus (HSV-1). HSV-1 is efficiently transported between synaptically connected neurons, and hence can spread rapidly through the nervous system which would be beneficial for expression in cells distant from the site of administration. However, HSV vectors present several problems, including instability of expression, eliciting an immune response, and reversion to wild-type. In an attempt to circumvent the difficulties inherent in the recombinant HSV vector, defective HSV vectors were employed as gene transfer vehicles within the nervous system. The defective HSV vector is a plasmid-based system, whereby a plasmid vector (termed an amplicon) is generated which contains the gene of interest and two cis-acting HSV recognition signals. These are the origin of DNA replication and the cleavage packaging signal. These sequences encode no HSV gene products. In the presence of HSV proteins provided by a helper virus, the amplicon is replicated and packaged into an HSV coat. This vector therefore expresses no viral gene products within the recipient cell, and recombination with or reactivation of latent viruses by the vector is limited due to the minimal amount of HSV DNA sequence present within the defective HSV vector genome. The major limitation of this system, however, is the inability to eliminate residual helper virus from the defective vector stock. The helper virus is often a mutant HSV which, like the recombinant vectors, can only replicate under permissive conditions in tissue culture. The continued presence of mutant helper HSV within the defective vector stock, however, presents problems which are similar to those enumerated above in regard to the recombinant HSV vector. This would therefore serve to limit the usefulness of the defective HSV vector for human applications. While HSV vectors of reduced toxicity and replication ability have been suggested, they can still mutate to a more dangerous form, or activate a latent virus, and, since the HSV does not integrate, achieving long-term expression would be difficult.

Lentivirus-based vectors have also been developed. Lentiviruses are complex retroviruses, which, in addition to the common retroviral genes gag, pol, and env, contain other genes with regulatory or structural function. The higher complexity enables the virus to modulate its life cycle, as in the course of latent infection. A typical lentivirus is the Human Immunodeficiency Virus (HIV), the etiologic agent of AIDS. Lentivirus vectors have shown potential upon strains having expanded tropism were discovered, including the transduction of cells of the CNS. Naldini et al., (1996) PNAS USA 93: 11382-11388. However, in view of the role of lentivirus in human diseases such as AIDS, important safety concerns remain. Adenoviral vectors have also been explored, but retention and expression of many adenovirus genes presents problems similar to those described with the HSV vector, particularly the problem of cytotoxicity to the recipient cell. In addition, recombinant adenovirus vectors often elicit immune responses which may serve to both limit the effectiveness of vector-mediated gene transfer and may provide another means for destruction of transduced cells. Finally, as with the HSV vectors, stability of long-term expression is currently unclear since there is no mechanism for specific viral integration in the genome of non-dividing host cells at high frequency.

Finally, a standard approach for somatic cell gene transfer, i.e., that of retroviral vectors, is not feasible for the brain, as retrovirally mediated gene transfer requires at least one cell division for integration and expression.

Thus, the gene therapy of most CNS diseases requires to transduce neurons that are terminally differentiated post-mitotic cells. Adeno-, lenti-, herpes simplex and adeno-associated virus vectors can deliver therapeutic genes to neurons with specific advantages and limits (reviewed in Kay et al., Nat. Med., 7:33-40, 2001; Vigna et al., J. Gene Med. 5:308-316, 2000; Kordower et al., Exp. Neurol., 160:1-16, 1999; Monahan et al., Mol. Med. Today 2000, 11:433-440; Peel et al., J. Neurosci Methods., 98:95-104, 2000; Lo et al., Hum. Gene Ther., 10:201-213, 1999; and Kaplitt et al., Nat. Genet., 8:148-154, 1994). However, direct stereotaxic injection of most viral vectors within anatomically distinct cerebral areas only allows neuron transduction close to the injection site, i.e. within tenths of mm of the penetrated zone.

Injections inside cerebral ventricules allows a global delivery along cerebrospinal fluid flow, but transduction of the therapeutic gene is restricted to ependymal and periventricular cells. See Ghodsi et al., Exp Neurol., 160:109-16, 1999; Driesse et al., Hum Gene Ther., 10:2347-54, 1999; Wang et al., Gene Ther., 4:1300-4, 1997; Oshiro et al., Cancer Gene Ther., 2:87-95, 1995. Systemic delivery of viral vectors with hyperosmotic mannitol to disrupt the bloodbrain barrier results in very limited transduction of dispersed neurons (Muldoon et al., Am J Pathol., 147:1840-51, 1995; Doran et al., Neurosurgery, 36(5):965-70, 1995). There is therefore a need for methods for the safe transduction of cells in the CNS allowing a gene of interest to be expressed not only in cells at the site of administration, but also in cells distant to the site of administration. There is also a need for methods for specifically transducing selected populations of cells in the CNS, particularly cell populations synaptically connected.

In one aspect, the present invention provides methods for delivering recombinant AAV (rAAV) virions carrying a transgene to neurons of a subject, for example a human, by administering rAAV vectors to a selected population of interconnected cells, preferably synaptically interconnected neurons. Preferably, the vectors are introduced to the central nervous system (CNS) via direct injection, most preferably via intracerebral injection. The inventors have demonstrated the ability of rAAV vectors to undergo anterograde transport across synapses. Also demonstrated is the long distance diffusion of a gene of interest to specific connected areas of the CNS, as well as the continued long term expression of a therapeutic polypeptide in cells of said distant regions of the CNS for at least 7-12 months.

The methods according to the invention of delivering a rAAV vector or more particularly a polypeptide to connected populations of cells of the CNS are expected to allow the treatment of many neurological disorders, especially motor neuron disorders and disorders characterized by neurodegeneration within specific connected neuron populations. The feasibility of such delivery have been evaluated in a model using mice deficient for the adrenoleukodystrophy (ALD) gene, encoding the ALD protein (ALDP), an intracellular nonsecreted protein from the ATP-binding cassette (ABC) family. ALD is a monogenic peroxisomal disorder characterized by diffuse demyelination within the CNS (Dubois-Dalcq et al., Trends Neurosci., 22:4-12, 1999; Moser, Brain., 120 (Pt 8):1485-508, 1997 August). The present inventors have followed and demonstrated the diffusion of the ALD gene in the CNS after administration of rAAV in the spinal cord, corpus callosum and pons of ALD deficient mice. Injection of rAAV bearing the ALD gene in the lumbar spinal cord resulted in the expression of ALDP in thalamus and colliculus neurons, revealing long distance anterograde transport of the therapeutic ALD gene. Comparable experiments in corpus callosum and pons of adult ALD mice and in the subventricular zone (SVZ) of ALD newborn ALD mice confirmed the long distance diffusion of tire ALD gene consistently to specific connected areas. Moreover, the long distance ALDP expression was still present 7-12 months after injection.

The invention encompasses methods for transducing a population of neurons with a rAAV vector; methods for transferring a foreign polynucleotide carried by a viral vector to a recipient cell; methods for expressing a nucleic acid sequence in a target population of neurons; methods for delivering recombinant AAV virions to a subject; and methods of the treatment of a subject suffering from a disease.

In one aspect, the methods of the invention comprise: selecting a first population and a second population of synaptically connected neurons, wherein a therapeutic polypeptide is to be expressed in said second population of neurons; administering rAAV virions to said first subpopulation of neurons of said subject, wherein said rAAV virions comprise a nucleic acid sequence encoding a therapeutic polypeptide.

In another aspect, the methods of the invention comprise: identifying a subject suspected of suffering from, or susceptible to developing, a condition characterized by the degeneration of, or a disorder in, at least a first and a second specific neuronal population that are synaptically connected; administering said rAAV virions such that rAAV virions are delivered to neurons of said subject, wherein said rAAV virions comprise a nucleic acid sequence encoding a therapeutic polypeptide.

As described further herein, preferably, the invention involves using rAAV virions capable of transducing synaptically connected neurons; or capable of being transported across a synapse between synaptically connected neurons; or achieving transduction of members of said synaptically connected population of neurons distant from the site of virion administration.

Said first and second populations of neurons are not required to be immediately adjacent, either in physical distance or connection distance, and may be separated by any number of synaptically connected neuron populations located between said first and second population. Said first and second populations of neurons are generally separated by at least one synapse, but may also be separated by at least 2, 3, 4, 5, or 10 synapses.

Particularly in the use of animal models, the methods of the invention optionally further comprise detecting the expression of said therapeutic polypeptide in a CNS cell of said subject; or detecting the transduction by said rAAV virions of a CNS cell of said subject.

In preferred embodiments, the methods of the invention allow selective targeting of neuron populations which are to be transduced with rAAV virions. Preferably, said rAAV virions transduce cells consisting essentially of neurons synaptically connected to one another.

In other embodiments of the methods of the invention, said first or second population of neurons comprise neurons of the CNS. In certain embodiments, said second population of neurons is a population of motor neurons.

Preferably, administration comprises direct intracerebral administration. More preferably, said intracerebral administration is by stereotactic microinjection.

As further described herein, the rAAV virions may comprise a nucleic acid sequence encoding any desired polypeptide, either secreted or non-secreted, and, preferably but not limited to a therapeutic polypeptide.

In further preferred embodiments, the invention relates to the treatment of disorders affecting populations of neurons. Encompassed are methods for treating or preventing a neurodegenerative disease in a subject comprising: providing a preparation comprising recombinant adeno-associated virus (rAAV) virions, wherein said virions comprise a nucleic acid sequence that is expressible in transduced cells to provide a therapeutic effect in the subject; and selecting a first population and a second population of synaptically connected neurons, wherein a therapeutic polypeptide is to be expressed in said second population of neurons; delivering the preparation to said first population of neurons of the subject, wherein the nucleic acid sequence is expressed to provide a therapeutic effect in the subject suitable for treating said neurodegenerative disease. Besides neurodegenerative disorders, said method can also be analogously applied to any other suitable CNS disorder, or any other situation wherein a transgene is to be expressed in a population of neurons.

In exemplary embodiments, the neurodegenerative disease is Alzheimer's disease. The rAAV preparation may be delivered to the corpus amygdaloideum of the subject and/or to the entorhinal cortex of the subject. The therapeutic polypeptide may be a polypeptide selected from the group consisting of: a polypeptide capable of inhibiting or reducing the formation of Aβ production; a polypeptide capable of modifying APP processing; a polypeptide capable of stimulating α-secretase cleavage activity; a polypeptide capable of inhibiting the β-secretase pathway; a polypeptide capable of inhibiting the γ-secretase pathway; and a polypeptide capable of inhibiting tau protein hyperphosphorylation.

Also encompassed are methods for treating or preventing a motor neuron disease in a subject comprising: providing a preparation comprising recombinant adeno-associated virus (rAAV) virions, wherein said virions comprise a nucleic acid sequence that is expressible in transduced cells to provide a therapeutic effect in the subject; and selecting a first population and a second population of synaptically connected neurons, wherein a therapeutic polypeptide is to be expressed in said second population of neurons; delivering the preparation to said first population of neurons of the subject, wherein the nucleic acid sequence is expressed to provide a therapeutic effect in the subject suitable for treating said a motor neuron disease.

In exemplary embodiments, the rAAV virions are delivered to the ruber nucleus, to the ventralis lateralis, or to the anterior nuclei of the thalamus. Said injection of rAAV virions to the ruber nucleus allows targetting of the lower motor neurons located along the spinal cord.

One example of a motor neuron disease which may be treated using the present methods is amyotrophic lateral sclerosis (ALS). In one example of the treatment of ALS, the therapeutic polypeptide is superoxide dismutase 1 (SOD1). In another example, the polypeptide is a polypeptide capable of inhibiting apoptotic cell death or a trophic factor.

In another example, the motor neuron disease is SMA. The therapeutic polypeptide comprised in said rAAV virions may be for example SMN2, a trophic factor or a polypeptide capable of decreasing glutamate toxicity.

Kennedy's disease (bulbospinal atrophy) is yet a further example of a motor neuron disease which may be treated using the present methods. In preferred examples, a therapeutic polypeptide expressed for the treatment of Kennedy's disease is a chaperone polypeptide, or a polypeptide capable of increasing chaperone polypeptide expression, a trophic factor, or a polypeptide capable of decreasing glutamate toxicity.

Further examples of motor neuron disease which may be treated using the present methods, such as paraplegia, will be readily appreciated by the person of skill in the art.

Also encompassed are the use of rAAV virions in the manufacture of a medicaments for use in a method of treating disease, preferably a condition characterized by the degeneration of, or a disorder in, at least a first and a second specific neuronal population that are synaptically connected. Also embodied is the use of rAAV virions carrying a transgene in the preparation of a medicament for the treatment of a disease in a subject, wherein a first population and a second population of synaptically connected neurons are selected and a therapeutic polypeptide is to be expressed in said second population of neurons; and a medicament comprising recombinant adeno-associated virus (rAAV) virions is delivered to said first population of neurons of the subject, wherein said virions comprise a nucleic acid sequence that is expressible in transduced cells to provide a therapeutic effect in the subject.

Further embodiments may comprise method for regulating the expression of a nucleic acid encoding a therapeutic polypeptide in a population of cells. In other embodiments, the methods according to the invention may be used for the treatment of tumors. In one example, a prodrug system can be used, for example a suicide gene therapy based approach wherein sensitivity to a compound is conferred on tumor cells.

These and other embodiments of the subject invention will readily occur to those of ordinary skill in the art in view of the disclosure herein.

### BRIEF DESCRIPTION OF THE DRAWINGS

FIGS. 1A and 1B are diagrams of the human brain showing neural connections between different areas of the brain cortex, the entorhinal cortex and the hippocampus. Studies have shown that the progression of neural lesions in Alzheimer's disease follows particular neural connections.

FIGS. 2A, 2B and 2C show the spatial progression of neurofibrillary tangles and amyloid deposits in Alzheimer's disease. In FIG. 2A, neurofibrillary tangles accumulate in the entorhinal cortex (2A, in blue). Then, in FIG. 2B, amyloid deposits and neurofibrillary tangles (2B, in green) are present in the entorhinal cortex and hippocampus whereas amyloid deposits are present in the associative areas of brain (2B, in yellow). In FIG. 2C, at a late stage, neurofibrillary tangles and amyloid deposits are present in most cortical areas (2C, in green). The primary cortex areas are the last involved brain regions where amyloid deposits accumulate (2C, in yellow).

FIG. 3A is a diagram showing efferent connections of the corpus amygdaloideum to the cerebral cortex. Delivery of rAAV to the corpus amygdaloideum can be used to target rAAV to various associative brain areas.

FIG. 3B is a diagram showing direct and indirect afferent connections to he hippocampus. Delivery of rAAV to the entorhinal cortex can be used to target rAAV to various associative brain areas (7, 9, 22, 46).

FIG. 4 is a diagram of the CNS showing components of the two-neuron pathway involved in motor neuron diseases, also indicated. Cell bodies of upper motor neurons in the primary motor cortex in the cerebral cortex project long axons to the spinal cord and brainstem, where they are in synaptic connection with lower motor neurons, which in turn project axons out through cranial and spinal nerves to synapses on muscle fibers of the head and body.

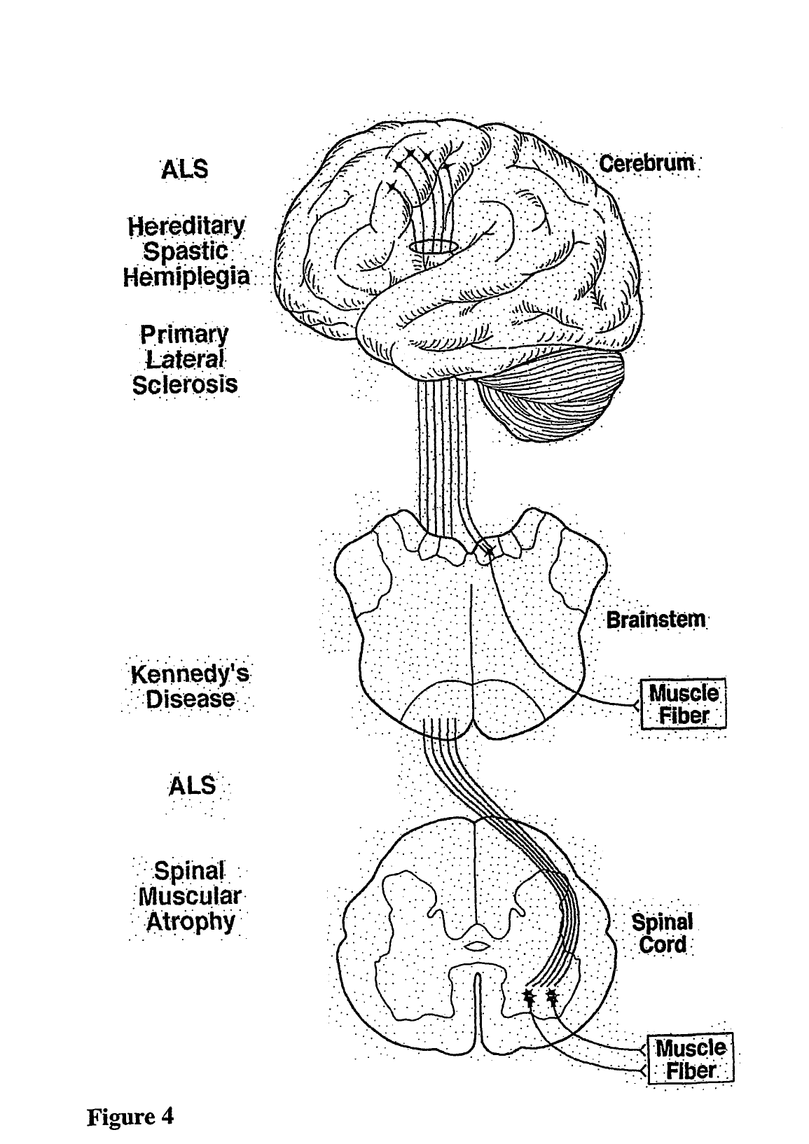

FIG. 5 is a diagram of the brain showing neuronal connections of the pyramidal system. rAAV vectors can advantageously be delivered to limited brain structure such as the ruber nucleus (9 and/or 10) which projects to scattered motor neurons in the spinal cord.

FIG. 6 is a diagram of the brain showing projections from the nucleus ventralis lateralis of the thalamus to the premotor cortex. Transducing neurons of the ventralis lateralis with rAAV vectors allows the transduction of a large number of motor neurons in the premotor cortex.

FIG. 7 is a diagram of the brain showing projections from the nucleus ventralis lateralis and nucleus medialis of the thalamus to the prefrontal cortex. Transducing neurons of the nucleus ventralis lateralis and nucleus medialis with rAAV vectors allows the transduction of a large number of motor neurons in the prefrontal cortex.

FIG. 8 shows the localization of ALDP positive cells in the brain of adult and newborn ALD mice after injection of PGK-hALD-AAV in corpus callosum, pons (adult mice) and subventricular zone (newborn mice). The distribution and density of ALDP positive cells in the injected hemisphere is indicated by dots. Identical results were obtained in two other adult ALD mice at 7 months and 4 other newborn ALD mice at 6 and 12 months. The localization of each brain section is indicated by horizontal lines in the left column. Injection sites are indicated by vertical arrows.

### DETAILED DESCRIPTION OF THE INVENTION

Until recently, most studies of viral based gene transfer into the CNS focused on retroviruses, lentiviruses, HSV-1 vectors and adenovirus. Geller et al., PNAS USA 87:1149-1153, 1990; Spaete et al., Cell 30:295-304, 1982; Martuza et al., Science 252:854-856, 1991; Davidson et al., Nat. Genetics 3:219-223, 1993; and Wilson et al., 5:501-519, 1994.

Also, for HSV vectors, see During et al., Science, 25:266(5189):1399-403, 1994 November; Kramm et al., Hum Gene Ther., 8:2057-68, 1997; Burton et al., Gene Yher. Mol. Biol., 5:1-17, 2000; Latchman et al., Biochem Soc. Trans., 27:847-51, 1999.

For adenovirus vectors, see Zou et al., Hum. Gene Ther., 12:181-19, 2001; Benihoud et al., Curr. Opin. Biotechnol., 10: 440-447, 1999; Barkats et al., Prog. Neurobiol., 55(4):333-41, 1998 July; Le Gal La Salle et al., Science, 259:988-9990, 1993; Akli et al., Nat Genet., 3:224-8, 1993.

For lentivirus see Kordower et al., Science, 27;290(5492):767-73, 2000 October; Naldini et al., Science, 272:263-267, 1996; Blomer et al., J. Virol., 71:6641-6649, 1997; Kordower et al., Exp. Neurol., 160:1-16, 1999.

Because of the inability of retrovirus to transduce nondividing postmitotic cells such as neurons and most dormant glial cells in the CNS, this virus system has largely been devoted to developing a gene therapy approach to malignant CNS tumors containing rapidly dividing cells. HSV-1 vectors have several features that are advantageous for gene transfer into post-mitotic CNS, yet application of such vectors to human disease is problematic because of their documented cytotoxicity and immunogenicity, potential for reversion to wild-type and unknown interactions with a host already harboring latent HSV-1. Like retrovirus, adenovirus appear to transduce mitotic glial cells preferentially in vivo and in vitro, and concerns have been raised about its cytotoxicity and immunogenicity, believed to be related in part to persistent expression of viral proteins.

Adeno-associated virus (AAV) based vectors are emerging as the leading candidates for use in gene therapy. AAV is a helper-dependent DNA parvovirus which belongs to the genus Dependovirus. AAV requires infection with an unrelated helper virus, either adenovirus, a herpesvirus or vaccinia, in order for a productive infection to occur. The helper virus supplies accessory functions that are necessary for most steps in AAV replication. AAV infects a broad range of tissue, and has not elicited the cytotoxic effects and adverse immune reactions in animal models that have been seen with other viral vectors. Moreover, helper-free virus stocks can be obtained which do not express any viral proteins, rendering an immune response less likely.

Because it can transduce nondividing tissue, AAV may be well adapted for delivering genes to the central nervous system (CNS). AAV vectors have been shown to transduce neurons, with no evidence of cytotoxicity (Freese et al., Epilepsia, 38(7):759-766, 1997). AAV vectors are reviewed in general in Monahan et al., Gene Therapy, 7:24-30, 2000. Furthermore, U.S. Pat. No, 5,677,158 described methods of making AAV vectors. AAV vectors containing therapeutic genes under the control of the cytomegalovirus (CMV) promoter have been shown to transduce mammalian brain and to have functional effects in models of disease. AAV vectors carrying transgenes have been described, for example, in For AAV vectors see Kaplitt et al., Nat. Genet., 8:148-154, 1994; Mandel et al., Proc. Natl. Acad. Sci., U.S.A., 94:14083-14088, 1997; Lo et al., Hum. Gene Ther., 10:201-21, 1999; Bankiewicz et al., Exp. Neurol., 164:2-14, 2000; Peel et al., J Neurosci Methods., 98:95-104, 2000; Bueler H., Biol. Chem., 380:613-22, 1999; Rabinowitz et al., Curr Opin Biotechnol., 9:470-5, 1998; Monahan et al., Mol. Med. Today 2000, 11:433-440. However, delivery of AAV vectors to the CNS has proven difficult. To date, many examples in the literature have shown that delivery of AAV vectors has resulted only in local transduction of cells at the site of injection.

The practice of the present invention will employ, unless otherwise indicated, conventional methods of virology, microbiology, molecular biology and recombinant DNA techniques within the skill of the art. Such techniques are explained fully in the literature. See, e.g., Sambrook, et al. Molecular Cloning: A Laboratory Manual (Current Edition); Current Protocols in Molecular Biology (F. M. Ausubel, et al. eds., current edition); DNA Cloning: A Practical Approach, vol. I & 11 (D. Glover, ed.); Oligonucleotide Synthesis (N. Gait, ed., Current Edition); Nucleic Acid Hybridization (B. Haines & S. Higgins, eds., Current Edition); Transcription and Translation (B. Hames & S. Higgins, eds., Current Edition); CR C Handbook of Parvoviruses, vol. I & 11 (P. Tij essen, ed.); Fundamental Virology, 2nd Edition, vol. I & 11 (B. N. Fields and D. M. Knipe, eds.) As used in this specification and the appended claims, the singular forms “a,” “an” and “the” include plural references unless the content clearly dictates otherwise.

Definitions

“Gene transfer” or “gene delivery” refers to methods or systems for reliably inserting foreign DNA into host cells. Such methods can result in transient expression of non-integrated transferred DNA, extrachromosomal replication and expression of transferred replicons (e.g., episomes), or integration of transferred genetic material into the genomic DNA of host cells. Gene transfer provides a unique approach for the treatment of acquired and inherited diseases. A number of systems have been developed for gene transfer into mammalian cells.

By “vector” is meant any genetic element, such as a plasmid, phage, transposon, cosmid, chromosome, virus, virion, etc., which is capable of replication when associated with the proper control elements and which can transfer gene sequences between cells. Thus, the term includes cloning and expression vehicles, as well as viral vectors.

By “recombinant virus” is meant a virus that has been genetically altered, e.g., by the addition or insertion of a heterologous nucleic acid construct into the particle.

By “AAV virion” is meant a complete virus particle, such as a wild-type (wt) AAV virus particle (comprising a linear, single-stranded AAV nucleic acid genome associated with an AAV capsid protein coat). In this regard, single-stranded AAV nucleic acid molecules of either complementary sense, e.g., “sense” or “antisense” strands, can be packaged into any one AAV virion and both strands are equally infectious.

A “recombinant AAV virion,” or “rAAV virion” is defined herein as an infectious, replication-defective virus based on the AAV virus—generally composed of an AAV protein shell, encapsidating a heterologous nucleotide sequence of interest which is flanked on both sides by AAV ITRs. A rAAV virion can be produced in a suitable host cell which has had an AAV vector, AAV helper functions and accessory functions introduced therein. In this manner, the host cell is rendered capable of encoding AAV polypeptides that are required for packaging the AAV vector (containing a recombinant nucleotide sequence of interest) into infectious recombinant virion particles for subsequent gene delivery.

The term “transduction” refers to the viral transfer of genetic material and its expression in a recipient cell.

The term “host cell” denotes, for example, microorganisms, yeast cells, insect cells, and mammalian cells, that can be, or have been, used as recipients of an AAV helper construct, an AAV vector plasmid, an accessory function vector, or other transfer DNA.

The term includes the progeny of the original cell which has been transfected. Thus, a “host cell” as used herein generally refers to a cell to which has been introduced an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “coding sequence” or a sequence which “encodes” a particular protein, is a nucleic acid sequence which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and even synthetic DNA sequences.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter region” is used herein in its ordinary sense to refer to a nucleic acid region comprising a DNA regulatory sequence, wherein the regulatory sequence is derived from a nucleic acid sequence which is capable of binding RNA polymerase and initiating transcription of a downstream (Y-direction) coding sequence.

“Operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

By “isolated” when referring to a nucleotide sequence, is meant that the indicated molecule is present in the substantial absence of other biological macromolecules of the same type. Thus, an “isolated nucleic acid molecule which encodes a particular polypeptide” refers to a nucleic acid molecule which is substantially free of other nucleic acid molecules that do not encode the subject polypeptide; however, the molecule may include some additional bases or moieties which do not deleteriously affect the basic characteristics of the composition. For the purpose of describing the relative position of nucleotide sequences in a particular nucleic acid molecule throughout the instant application, such as when a particular nucleotide sequence is described as being situated “upstream,” “downstream,” relative to another sequence, it is to be understood that it is the position of the sequences in the “sense” or “coding” strand of a DNA molecule that is being referred to as is conventional in the art.

A “gene” refers to a polynucleotide containing at least one open reading frame that is capable of encoding a particular polypeptide or protein after being transcribed or translated. Any of the polynucleotide sequences described herein may be used to identify larger fragments or full-length coding sequences of the genes with which they are associated. Methods of isolating larger fragment sequences are know to those of skill in the art.

As used herein, “synaptically connected” neurons refers to neurons which are in communication with one another via a synapse. A synapse is a zone of a neuron specialized for signal transfer. Synapses can be characterized by their ability to act as a region of signal transfer as well as by the physical proximity at the synapse between two neurons. Signalling can be by electrical or chemical means.

“Delivering” refers to any means and/or method of providing an agent. In the context of the CNS, “direct delivery” refers to local delivery, generally by physically intervening to place or inject an agent at a particular location in the CNS.

As used herein, a “population” of neurons refers to a plurality of neurons wherein said the members of the population of neurons are distinguishable from members of other populations of neurons by a common characteristic. Said characteristic is not limited to but may include a common localization in the central nervous system and/or a common biological function. Members of a “population” may therefore be distinguished based for example on localization, functional assays, or any other suitable means of identification, including expression of specific biomarkers. The term population of neurons encompasses a population of CNS neurons as well as a population of peripheral nervous system neurons.

The term “central nervous system” or “CNS” includes all cells and tissue of the brain and spinal cord. Thus, the term includes, but is not limited to, neuronal cells, glial cells, astrocytes, cerebrospinal fluid (CSF), interstitial spaces, and the like.

The terms “subject”, “individual” or “patient” are used interchangeably herein and refer to a vertebrate, preferably a mammal, and more preferably a human. Mammals include, but are not limited to, murines, simians, humans, farm animals, sport animals and pets.

An “effective amount” is an amount sufficient to effect beneficial or desired results. An effective amount can be administered in one or more administrations, applications or dosages.

### GENERAL OVERVIEW OF THE INVENTION

Central to the present invention is the development of methods which allow for delivery of AAV vectors into the CNS of animal such that the vector can transduce cells of the CNS located outside of the local area of injection. The present invention not only provides vectors that can transduce cells in situations where diffuse delivery of a nucleic acid is required, but also allows vector proliferation to be controllable by selecting populations of interconnected cells to be transduced.

Previously, researchers have had little success delivering viral vectors to the brain using AAV vectors. A large body of literature demonstrates that delivery using AAV vectors with direct intracerebral delivery results in only local transduction of cells. Such delivery would not be adequate for the treatment of many neurodegenerative disorders in which a diffuse gene therapy is required. While other methods have used e.g. convection enhanced delivery or multiple sites of AAV vector administration in order to achieve broader delivery of vector, delivery nevertheless remains inadequate in the specific populations of cells in which expression of a therapeutic gene is to be obtained. Moreover, broad expression throughout the nervous system would in most cases not be desirable in view of toxicity or adverse effects of transgene expression.

The present invention thus provides methods of transducing selected populations of neurons with AAV vectors. This is made possible by AAV vectors capable of being transported across synapses between connected neurons. Advantages of the invention, include, but are not limited to (i) delivery of viral vectors to cells of the CNS distant from the site of injection; (ii) expression of nucleic acids (e.g., transgenes), including nucleic acids encoding non-secreted proteins carried by the viral vectors; (iii) and targeted transduction by viral vectors of neurons which are synaptically connected. The present invention enables treatments for a large number of disorders in which delivery of a transgene to neurons is required, including preferably neurodegernative disorders such as Alzheimers' disease and other motor neuron disorders such as ALS.

Construction of Viral Vectors

Gene delivery vehicles useful in the practice of the present invention can be constructed utilizing methodologies well known in the art of molecular biology (see, for example, Ausubel or Maniatis, supra). Typically, viral vectors carrying transgenes are assembled from polynucleotides encoding the transgene(s), suitable regulatory elements and elements necessary for production of viral proteins which mediate cell transduction. For example, in a preferred embodiment, adeno-associated viral (AAV) vectors are employed.

General Methods

A preferred method of obtaining the nucleotide components of the viral vector is PCR. General procedures for PCR are taught in MacPherson et al., PCR: A PRACTICAL APPROACH (IRL Press at Oxford University Press, (1991)). DNA fragments can then be ligated together as appropriate. Polynucleotides are inserted into vector genomes using methods well known in the art. For example, insert and vector DNA can be contacted, under suitable conditions, with a restriction enzyme to create complementary or blunt ends on each molecule that can pair with each other and be joined with a ligase. Alternatively, synthetic nucleic acid linkers can be ligated to the termini of a polynucleotide. These synthetic linkers can contain nucleic acid sequences that correspond to a particular restriction site in the vector DNA.

AAV Expression Vectors

Preferably, viral vectors are AAV vectors. By an “AAV vector” is meant a vector derived from an adeno-associated virus serotype, including without limitation, AAV-1, AAV-2, AAV-3, AAV-4, AAV-5, AAV6, etc. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. AAV expression vectors are constructed using known techniques to at least provide as operatively linked components in the direction of transcription, control elements including a transcriptional initiation region, the DNA of interest and a transcriptional termination region.

The control elements are selected to be functional in a mammalian cell. The resulting construct which contains the operatively linked components is bounded (5′ and Y) with functional AAV ITR sequences. By “adeno-associated virus inverted terminal repeats” or “AAV ITRs” is meant the art-recognized regions found at each end of the AAV genome which function together in cis as origins of DNA replication and as packaging signals for the virus. AAV ITRS, together with the AAV rep coding region, provide for the efficient excision and rescue from, and integration of a nucleotide sequence interposed between two flanking ITRs into a mammalian cell genome. The nucleotide sequences of AAV ITR regions are known. See, e.g., Kotin, R. M., Human Gene Therapy, 5:793-801, 1994; Berns, K I “Parvoviridae and their Replication” in Fundamental Virology, 2nd Edition, (B. N. Fields and D. M. Knipe, eds.) for the AAV-2 sequence. As used herein, an “AAV ITR” need not have the wild-type nucleotide sequence depicted, but may be altered, e.g., by the insertion, deletion or substitution of nucleotides. Additionally, the AAV ITR may be derived from any of several AAV serotypes, including without limitation, AAV-1, AAV-2, AAV-3, AAV-4, AAV-5, AAV6, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell. Additionally, AAV ITRs may be derived from any of several AAV serotypes, including without limitation, AAV-1, AAV-2, AAV-3, AAV-4, AAV 5, AAV6, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell. Suitable DNA molecules for use in AAV vectors will be less than about 5 kilobases (kb) in size and will include, for example, a gene that encodes a protein that is defective or missing from a recipient subject or a gene that encodes a protein having a desired biological or therapeutic effect.

Particularly preferred are vectors derived from AAV serotypes having tropism for and high transduction efficiencies in cells of the mammalian CNS, particularly neurons. A review and comparison of transduction efficiencies of different serotypes is provided in Davidson et al., PNAS USA, 97(7):3428-3432, 2000. In one preferred example, AAV2 based vectors have been shown to direct long-term expression of transgenes in CNS, preferably transducing neurons. In other nonlimiting examples, preferred vectors include vectors derived from AAV4 and AAV5 serotypes, which have also been shown to transduce cells of the CNS (Davidson et al, supra).

The selected nucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the phophoglycerate kinase (PKG) promoter, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), rous sarcoma virus (RSV) promoter, synthetic promoters, hybrid promoters, and the like. In addition, sequences derived from nonviral genes, such as the murine metallothionein gene, will also find use herein. Such promoter sequences are commercially available from, e.g., Stratagene (San Diego, Calif.). For purposes of the present invention, both heterologous promoters and other control elements, such as CNS-specific and inducible promoters, enhancers and the like, will be of particular use. Examples of heterologous promoters include the CMV promoter. Examples of CNS-specific promoters include those isolated from the genes from myelin basic protein (MBP), glial fibrillary acid protein (GFAP), and neuron specific enolase (NSE). Examples of inducible promoters include DNA responsive elements for ecdysone, tetracycline, hypoxia and aufin.

The AAV expression vector which harbors the DNA molecule of interest bounded by AAV ITRs, can be constructed by directly inserting the selected sequence(s) into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publications Nos. WO 92/01070 (published 23 Jan. 1992) and WO 93/03769 (published 4 Mar. 1993); Lebkowski et al., Molec. Cell. Biol. 8:3988-3996, 1988; Vincent et al., Vaccines 90 (Cold Spring Harbor Laboratory Press), 1990; Carter, B. J., Current Opinion in Biotechnology, 3:533-539, 1992; Muzyczka, N., Current Topics in Microbiol. and Immunol., 158:97-129, 1992; Kotin, R. M., Human Gene Therapy 5:793-801, 1994; Shelling and Smith, Gene Therapy:165-169, 1994; and Zhou et al., J Exp. Med. 179:1867-1875, 1994. Alternatively, AAV ITRs can be excised from the viral genome or from an AAV vector containing the same and fused 5′ and 3′ of a selected nucleic acid construct that is present in another vector using standard ligation techniques, such as those described in Sambrook et al., supra. AAV vectors which contain ITRs have been described in, e.g., U.S. Pat. No. 5,139,941. In particular, several AAV vectors are described therein which are available from the American Type Culture Collection (“ATCC”) under Accession Numbers 53222, 53223, 53224, 53225 and 53226.

Additionally, chimeric genes can be produced synthetically to include AAV ITR sequences arranged 5′ and 3′ of one or more selected nucleic acid sequences. Preferred codons for expression of the chimeric gene sequence in mammalian CNS cells can be used. The complete chimeric sequence is assembled from overlapping oligonucleotides prepared by standard methods. See, e.g., Edge, Nature, 292:756, 1981; Nambair et al., Science, 223:1299, 1984; Jay et al., J. Biol. Chem., 259:6311, 1984. In order to produce rAAV virions, an AAV expression vector is introduced into a suitable host cell using known techniques, such as by transfection. A number of transfection techniques are generally known in the art. See, e.g., Graham et al., Virology, 2:456, 1973, Sambrook et al. (1989) Molecular Cloning, a laboratory manual, Cold Spring Harbor Laboratories, New York, Davis et al. (1986) Basic Methods in Molecular Biology, Elsevier, and Chu et al., Gene, 13:197, 1981. Particularly suitable transfection methods include calcium phosphate co-precipitation (Graham et al., Virol., 52:456 467, 1973), direct micro-injection into cultured cells (Capecchi, M. R., Cell, 22:479-488, 1980), electroporation (Shigekawa et al., BioTechniques, 6:742-751, 1988), liposome mediated gene transfer (Mannino et al., BioTechniques, 6:682 690, 1988), lipid-mediated transduction (Felgner et al., Proc. Natl. Acad. Sci., USA, 84:7413-7417, 1987), and nucleic acid delivery using high-velocity microprojectiles (Klein et al., Nature, 327:70-73, 1987).

For the purposes of the invention, suitable host cells for producing rAAV virions include microorganisms, yeast cells, insect cells, and mammalian cells, that can be, or have been, used as recipients of a heterologous DNA molecule. The term includes the progeny of the original cell which has been transfected. Thus, a “host cell” as used herein generally refers to a cell which has been transfected with an exogenous DNA sequence. Cells from the stable human cell line, 293 (readily available through, e.g., the American Type Culture Collection under Accession Number ATCC CRL 15 73) are preferred in the practice of the present invention. Particularly, the human cell line 293 is a human embryonic kidney cell line that has been transformed with adenovirus type-5 DNA fragments (Graham et al., J. Gen. Virol., -36:59, 1977), and expresses the adenoviral Ela and Elb genes (Aiello et al., Virology, 94:460, 1979). The 293 cell line is readily transfected, and provides a particularly convenient platform in which to produce rAAV virions.

AAV Helper Functions

Host cells containing the above-described AAV expression vectors must be rendered capable of providing AAV helper functions in order to replicate and encapsidate the nucleotide sequences flanked by the AAV ITRs to produce rAAV virions. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs, namely the rep and cap coding regions, or functional homologues thereof. The Rep expression products have been shown to possess many functions, including, among others: recognition, binding and nicking of the AAV origin of DNA replication; DNA helicase activity; and modulation of transcription from AAV (or other heterologous) promoters. The Cap expression products supply necessary packaging functions. AAV helper functions are used herein to complement AAV functions in trans that are missing from AAV vectors. The term “AAV helper construct” refers generally to a nucleic acid molecule that includes nucleotide sequences providing AAV functions deleted from an AAV vector which is to be used to produce a transducing vector for delivery of a nucleotide sequence of interest. AAV helper constructs are commonly used to provide transient expression of AAV rep and/or cap genes to complement missing AAV functions that are necessary for lytic AAV replication; however, helper constructs lack AAV ITRs and can neither replicate nor package themselves. AAV helper constructs can be in the form of a plasmid, phage, transposon, cosmid, virus, or virion. A number of AAV helper constructs have been described, such as the commonly used plasmids pAAV/Ad and pIM29+45 24 which encode both Rep and Cap expression products. See, e.g., Samulski et al., J Virol., 63:3822-3828, 1989; and McCarty et al., J. Virol., 65:2936-2945, 1991. A number of other vectors have been described which encode Rep and/or Cap expression products. See, e.g., U.S. Pat. No. 5,139,941. By “AAV rep coding region” is meant the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. For a description of the AAV rep coding region, see, e.g., Muzyczka, N., Current Topics in Microbiol. and Immunol. 158:97-129, 1992; and Kotin, R. M., Human Gene Therapy, 5:793-801, 1994. Suitable homologues of the AAV rep coding region include the human herpesvirus 6 (HHV-6) rep gene which is also known to mediate AAV-2 DNA replication (Thomson et al., Virology 204:304-311, 1994). By “AAV cap coding region” is meant the art-recognized region of the AAV genome which encodes the capsid proteins VP1, VP2, and VP3, or functional homologues thereof. These Cap expression products supply the packaging functions which are collectively required for packaging the viral genome. For a description of the AAV cap coding region, see, e.g., Muzyczka, N. and Kotin, R. M. (supra). AAV helper functions are introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves.

These constructs can be in the form of a plasmid, phage, transposon, cosmid, virus, or virion. A number of AAV helper constructs have been described, such as the commonly used plasmids pAAV/Ad and pIM29+45 which encode both Rep and Cap expression products. See, e.g., Samulski et al., J. Virol., 63:3822-3828, 1989; and McCarty et al., J. Virol., 65:2936-2945, 1991. A number of other vectors have been described which encode Rep and/or Cap expression products. See, e.g., U.S. Pat. No. 5,139,941. Both AAV expression vectors and AAV helper constructs can be constructed to contain one or more optional selectable markers. Suitable markers include genes which confer antibiotic resistance or sensitivity to, impart color to, or change the antigenic characteristics of those cells which have been transduced with a nucleic acid construct containing the selectable marker when the cells are grown in an appropriate selective medium. Several selectable marker genes that are useful in the practice of the invention include the hygromycin B resistance gene (encoding Aminoglycoside phosphotranferase (APH)) that allows selection in mammalian cells by conferring resistance to G418 (available from Sigma, St. Louis, Mo.). Other suitable markers are known to those of skill in the art.

AAV Accessory Functions

The host cell (or packaging cell) must also be rendered capable of providing non AAV derived functions, or “accessory functions,” in order to produce rAAV virions. Accessory functions are non AAV derived viral and/or cellular functions upon which AAV is dependent for its replication. Thus, accessory functions include at least those non AAV proteins and RNAs that are required in AAV replication, including those involved in activation of AAV gene transcription, stage specific AAV mRNA splicing, AAV DNA replication, synthesis of Cap expression products and AAV capsid assembly. Viral based accessory functions can be derived from any of the known helper viruses. Particularly, accessory functions can be introduced into and then expressed in host cells using methods known to those of skill in the art.

Commonly, accessory functions are provided by infection of the host cells with an unrelated helper virus. A number of suitable helper viruses are known, including adenoviruses; herpesviruses such as herpes simplex virus types 1 and 2; and vaccinia viruses.

Nonviral accessory functions will also find use herein, such as those provided by cell synchronization using any of various known agents. 26 See, e.g., Buller et al., J Virol. 40:241-247, 1981; McPherson et al., Virology, 147:217-222, 1985; Schlehofer et al., Virology, 152:110-117, 1986. Alternatively, accessory functions can be provided using an accessory function vector. Accessory function vectors include nucleotide sequences that provide one or more accessory functions. An accessory function vector is capable of being introduced into a suitable host cell in order to support efficient AAV virion production in the host cell. Accessory function vectors can be in the form of a plasmid, phage, transposon or cosmid. Accessory vectors can also be in the form of one or more linearized DNA or RNA fragments which, when associated with the appropriate control elements and enzymes, can be transcribed or expressed in a host cell to provide accessory functions. See, for example, International Publication No. WO 97/17548, published May 15, 1997.

Nucleic acid sequences providing the accessory functions can be obtained from natural sources, such as from the genome of an adenovirus particle, or constructed using recombinant or synthetic methods known in the art. In this regard, adenovirus-derived accessory functions have been widely studied, and a number of adenovirus genes involved in accessory functions have been identified and partially characterized. See, e.g., Carter, B. J. (1990), “Adeno Associated Virus Helper Functions,” in CR C Handbook of Parvoviruses, vol. I (P. Tijssen, ed.), and Muzyczka, N. (1992), Curr. Topics. Microbiol. and Immun. 15 8:97-129. Specifically, early adenoviral gene regions E I a, E2a, E4, VAI RNA and, possibly, Elb are thought to participate in the accessory process. Jailik et al., Proc. Natl. Acad. Sci., USA 78:1925-1929, 1981. Herpesvirus-derived accessory functions have been described. See, e.g., Young et al., Prog. Med. Virol., 25:113, 1979. Vaccinia virus-derived accessory functions have also been described. See, e.g., Carter, B. J. (1990), supra., Schlehofer et al. (1986), Virology 152:110 117. As a consequence of the infection of the host cell with a helper virus, or transfection of the host cell with an accessory function vector, accessory functions are expressed which transactivate the AAV helper construct to produce AAV Rep and/or Cap proteins.

The Rep expression products excise the recombinant DNA (including the DNA of interest) from the AAV expression vector. The Rep proteins also serve to duplicate the AAV genome. The expressed Cap proteins assemble into capsids, and the recombinant AAV genome is packaged into the capsids. Thus, productive AAV replication ensues, and the DNA is packaged into rAAV virions. Following recombinant AAV replication, rAAV virions can be purified from the host cell using a variety of conventional purification methods, such as CsCl gradients.

Further, if infection is employed to express the accessory functions, residual helper virus can be inactivated, using known methods. For example, adenovirus can be inactivated by heating to temperatures of approximately 600° C. for, e.g., 20 minutes or more. This treatment effectively inactivates only the helper virus since AAV is extremely heat stable while the helper adenovirus is heat labile. The resulting rAAV virions are then ready for use for DNA delivery to the CNS (e.g., cranial cavity) of the subject.

Nucleic Acids

As will be appreciated by the skilled person, according to the invention, the rAAV vectors can comprise any suitable nucleic acid sequence which is to be expressed in a desired cell. In one aspect, a nucleic acid sequence may serve to express a nucleic acid acting directly on a biological target, such as in an antisense or ribozyme treatment. In other aspects, said nucleic acid sequence may encode a polypeptide. As used herein, the terms peptide and polypeptides are used interchangeably, as polypeptides of essentially any length may be used in accordance with the present invention. Polypeptides may be full-length polypeptides or fragments thereof suitable for a particular application (e.g. capable of restoring a biological activity, inhibiting a biological activity). Polypeptides may be secreted or non-secreted polypeptides.

Non-limiting examples of nucleic acids that can be expressed include nucleic acids encoding neuropeptides, neurotransmitters, enzymes involved in biosynthesis, proteins involved in intracellular signalling pathways, and receptors, for example postsynaptic receptors. For example, viral vectors have been developed encoding enzymes responsible for dopamine biosynthesis (Freese et al., Epilepsia 38 (7):759-766) and the GluR6 excitatory amino acid receptor subtype (Bergold et al., 1993, PNAS USA 90:6165-6169, 1997). In certain applications, nucleic acids may allow detection of virions and/or detection of transgene expression. Nucleic acids may encode detectable marker polypeptides, such as a fluorescent protein (ex. GFP) or another detectable polypeptide such as β-galactosidase, or any polypeptide allowing synaptically connected neurons to be traced e.g. in a model organism. Other non-limiting examples of genes suitable for use according to the invention include anti-apoptotic genes such as bcl-2, interleukin-1 converting enzyme, crmA, bcl-x1, FLIP, survivin, IAP, ILP; genes which provides target cells, preferably tumor cells, with enhanced susceptibility to a selected cytotoxic agent, such as the herpes simplex virus thymidine kinase (HSV-tk), cytochrome P450, human deoxycytidine kinase, and bacterial cytosine deaminase genes (See also Springer and Niculescu-Duvaz, J. Clin. Invest., 105:1161-1167, 2000). Also included are polypeptides which reduce glutamate toxicity, and polypeptides with act as calcium buffers or binding protein such as calbindin. Also encompassed are polypeptides capable of inhibiting the activity of an enzyme. For example, encompassed in Alzheimer's disease are a polypeptide capable of inhibiting or reducing the formation of Aβ production, a polypeptide capable of modifying APP processing, a polypeptide capable of stimulating or generally increasing α-secretase cleavage activity, a polypeptide capable of inhibiting the β-secretase pathway, a polypeptide capable of inhibiting the γ-secretase pathway, or a polypeptide capable of inhibiting tau protein hyperphosphorylation.

Other examples of nucleic acids that can be used with the present invention include nucleic acids coding for growth factors or neurotrophic factors, including but not limited to genes encoding: acidic fibroblast growth factor (aFGF; FGF-1); glial cell line-derived neurotrophic factor; brain-derived neurotrophic factor; nerve growth factor; TGF-α, EGF, extracellular matrix proteins (collagens, fibronectins, integrins); ornithine amino transferase; prostaglandin synthesis regulation proteins; trabecular meshwork proteins; NT-3, NT-4/5; hypoxanthine phosphoribosyltransferase; tyrosine hydroxylase, prostaglandin receptors, catalase and glutathione peroxidase; sequences encoding interferons, lymphokines, cytokines and antagonists thereof such as tumor necrosis factor (TNF), CD4 specific antibodies, and TNF or CD4 receptors; sequences encoding GABA receptor isoforms, the GABA synthesizing enzyme glutamic acid decarboxylase (GAD), calcium dependent potassium channels or ATP-sensitive potassium channels; and sequences encoding thymidine kinase. Also envisioned are sequences encoding antisense nucleic acids. Other examples of polypeptides that can be encoded include dopadecarboxylase, cell adhesion molecules, interleukin-1.beta.; superoxide dismutase, basic fibroblast growth factor, ciliary neurotrophic factor and neurotransmitter receptors.

Nucleotide sequences encoding these polypeptides are known to those of skill in the art. For example, Abraham et al., Science 233:545, 1986, disclose the nucleotide sequence of bovine bFGF, while the nucleotide sequence of human bFGF is disclosed by Abraham et al., EMBO J. 5:2523, 1986. Mergia et al., Biochem. Biophys. Res. Commun., 164:1121, 1989, provide the nucleotide sequence of the human aFGF gene. The nucleotide sequence of the rat glial cell line-derived neurotrophic factor is described by Springer et al., Exp. Neurol., 131:47, 1995. Maisonpeirre et al., Genomics 10:558, 1991, provide the nucleotide sequences of human and rat brain-derived neurotrophic factor, while Arab et al., Gene, 185:95, 1997, disclose the amino acid sequence of bovine brain-derived neurotrophic factor. Rat ciliary neurotrophic factor is described by Stocki et al., Nature 342:920, 1989. The nucleotide sequence of the human ciliary neurotrophic factor gene is disclosed by Negro et al., Eur. J. Biochem., 201:289, 1991, Lin et al., Science, 246:1023, 1989, and by Lam et al., Gene, 102:271, 1991. Ulrich et al., Nature, 303:821, 1983, provide a comparison of human and murine coding regions of beta-nerve growth factor genes. The nucleotide sequence of bovine interleukin-.beta.1 is disclosed by Leong et al., Nucl. Acids Res., 16:9054, 1988, while Bensi et al., Gene, 52:95, 1987, provide the nucleotide sequence of the human interleukin-1.beta. gene.

DNA molecules encoding such polypeptides can be obtained by screening cDNA or genomic libraries with polynucleotide probes having nucleotide sequences based upon known genes. Standard methods are well-known to those of skill in the art. See, for example, Ausubel et al. (eds.), SHORT PROTOCOLS IN MOLECULAR BIOLOGY, 3rd Edition, pages 2-1 to 2-13 and 5-1 to 5-6 (John Wiley & Sons, Inc. 1995).

Alternatively, DNA molecules encoding growth factors can be obtained by synthesizing DNA molecules using mutually priming long oligonucleotides. See, for example, Ausubel et al. (eds.), CURRENT PROTOCOLS IN MOLECULAR BIOLOGY, pages 8.2.8 to 8.2.13 (1990). Also, see Wosnick et al., Gene, 60:115, 1987; and Ausubel et al. (eds.), SHORT PROTOCOLS IN MOLECULAR BIOLOGY, 3rd Edition, pages 8-8 to 8-9 (John Wiley & Sons, Inc. 1995). Established techniques using the polymerase chain reaction provide the ability to synthesize DNA molecules at least two kilobases in length. Adang et al., Plant Molec. Biol., 21:1131, 1993; Bambot et al., PCR Methods and Applications, 2:266, 1993; Dillon et al., “Use of the Polymerase Chain Reaction for the Rapid Construction of Synthetic Genes,” in METHODS IN MOLECULAR BIOLOGY, Vol. 15: PCR PROTOCOLS: CURRENT METHODS AND APPLICATIONS, White (ed.), pages 263-268, (Humana Press, Inc. 1993); Holowachuk et al., PCR Methods Appl., 4:299, 1995.

A method for constructing an rAAV vector that expresses a foreign gene is further detailed in Example 1 herein, where a vector was produced that expresses a hALD gene under the control of the PKG gene promoter.

Delivery of Viral Vectors

As described herein, the invention relates to the delivery of recombinant AAV virions to a subject comprising administering an rAAV virion composition to first population of neurons such that a second population of neurons synaptically connected thereto, and in which a therapeutic polypeptide is to be expressed, are transduced by said rAAV virions. In this respect, methods of delivery of viral vectors to said first population of neurons includes generally any method suitable for delivery AAV to the neurons such that at least a portion of cells of a selected synaptically connected cell population is transduced. The vector may be delivered to any cells of the central nervous system, cells of the peripheral nervous system, or both. Generally, the vector will be delivered to the cells of the central nervous system, including for example cells of the spinal cord, brainstem (medulla, pons, and midbrain), cerebellum, diencephalon (thalamus, hypothalamus), telencephalon (corpus striatum, cerebral cortex, or, within the cortex, the occipital, temporal, parietal or frontal lobes), or combinations thereof, or preferably any suitable subpopulation thereof. Further preferred sites for delivery include the ruber nucleus, corpus amygdaloideum, entorhinal cortex and neurons in ventralis lateralis, or to the anterior nuclei of the thalamus.

In preferred methods, delivery methods comprise direct intracerebral delivery. To deliver the vector specifically to a particular region and to a particular population of cells of the CNS, the rAAV may be administered by stereotaxic microinjection (as exemplified in Example 4). For example, patients will have the stereotactic frame base fixed in place (screwed into the skull). The brain with stereotactic frame base (MRI-compatible with fiducial markings) will be imaged using high resolution MRI. The MRI images will then be transferred to a computer which runs stereotactic software. A series of coronal, sagittal and axial images will be used to determine the target (site of AAV vector injection) and trajectory. The software directly translates the trajectory into 3 dimensional coordinates appropriate for the stereotactic frame. Burr holes are drilled above the entry site and the stereotactic apparatus positioned with the needle implanted at the given depth. The AAV vector will then be injected at the target sites. Since the AAV vector will integrate into the target cells, rather than producing viral particles, the subsequent spread of the vector will be minor, and mainly a function of passive diffusion from the site of injection and of course the desired transsynaptic transport, prior to integration. The degree of diffusion may be controlled by adjusting the ratio of vector to fluid carrier.

Additional routes of administration may also comprise local application of the vector under direct visualization, e.g., superficial cortical application, or other non-stereotactic application. The vector may generally be delivered intrathecally, for specific applications.

The target cells of the vectors of the present invention are cells of the central or peripheral nervous systems of a mammal. Preferably, the cells are part of a living mammal at the time the vector is delivered of the cell. The mammal may be at any stage of development at the time of delivery, e.g., embryonic, fetal, infantile, juvenile or adult. Furthermore, the target CNS cells may be essentially from any source, including human cells, or cells of other mammals, especially nonhuman primates and mammals of the orders Rodenta (mice, rats, rabbit, hamsters), Carnivora (cats, dogs), and Arteriodactyla (cows, pigs, sheep, goats, horses) as well as any other non-human system (e.g. zebrafish model system) which may be useful as biological models of disease.

Preferably, the method of the invention comprises intracerebral administration. However, other known delivery methods may also be adapted in accordance with the invention. For example, for a more widespread distribution of the vector across the CNS, it may be injected into the cerebrospinal fluid, e.g., by lumbar puncture. To direct the vector to the peripheral nervous system, it may be injected into the spinal cord or into the peripheral ganglia, or the flesh (subcutaneously or intramuscularly) of the body part of interest. In certain situations the vector can be administered via an intravascular approach. For example, the vector can be administered intra-arterially (carotid) in situations where the blood-brain barrier is disturbed. Such conditions include cerebral infarcts (strokes) as well as some brain tumors. Moreover, for more global delivery, the vector can be administered during the “opening” of the blood-brain barrier achieved by infusion of hypertonic solutions including mannitol. Of course, with intravenous delivery, the user must be able to tolerate the delivery of the vector to cells other than those of the nervous system.

Pharmaceutical Compositions

As discussed above, for in vivo delivery, the rAAV virions will be formulated into pharmaceutical compositions and will generally be administered parenterally. The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in REMINGTON's PHARMACEUTICAL SCIENCES (Mack Pub. Co., N.J., 1991). As is apparent to those skilled in the art in view of the teachings of this specification, an effective amount of viral vector which must be added can be empirically determined.

Administration can be effected in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician. It should be understood that more than one transgene could be expressed by the delivered viral vector.

Alternatively, separate vectors, each expressing one or more different transgenes, can also be delivered to the CNS as described herein. Furthermore, it is also intended that the viral vectors delivered by the methods of the present invention be combined with other suitable compositions and therapies.

Pharmaceutical compositions will comprise sufficient genetic material to produce a therapeutically effective amount of the protein of interest, i.e., an amount sufficient to reduce or ameliorate symptoms of the disease state in question or an amount sufficient to confer the desired benefit. In animal models for gene transfer or of disease, or in cell culture-based applications, effective amount of the exogenous nucleic acid composition is such as to produce the desired effect in a host that can be monitored using several end-points known to those skilled in the art. Effective gene transfer of an exogenous nucleic acid to a host cell is confirmed by evidence of the transferred nucleic acid or expression of the exogenous nucleic acid within the host (e.g., using the polymerase chain reaction in conjunction with sequencing, Northern or Southern hybridizations, or transcription assays to detect the nucleic acid in host cells, or using immunoblot analysis, antibody-mediated detection, mRNA or protein half-life studies, or particularized assays to detect protein or polypeptide encoded by the transferred nucleic acid, or impacted in level or function due to such transfer). One such particularized assay includes the Western immunoassay for the detection of the protein encoded by the exogenous nucleic acid. These methods described herein are by no means all-inclusive, and further methods to suit the specific application will be apparent to the ordinary skilled artisan.

Treatment of CNS Disorders

In accordance with the methods of the present invention, the rAAV vectors expressing therapeutic transgenes can be used to treat, arrest, or prevent practically any CNS disorder which can be ameliorated by providing therapeutic proteins or polypeptides. Particularly advantageously, the methods and vectors according to the invention are used for the treatment of disorders affecting synaptically connected neurons. Also envisioned is the treatment of cells in proximity to said neurons such that a therapeutic protein expressed by a vector is delivered to said cells. In preferred embodiments, the methods of the invention are used for the treatment of neurodegenerative disorders and motor neuron diseases.

As discussed further herein, currently available methods using AAV-based vectors, particularly those involving direct local injection, either do not provide for diffusion or transport of vectors outside of the local area of administration, or do not allow preferential delivery to selected populations of cells. The present methods, however, allow for the treatment of disorders where a therapeutic polypeptide is to be expressed in a neuronal population located at distance from the site of injection, but also preferably not throughout the entire CNS. Furthermore, unlike ventricular delivery the present methods allows transduction of cells other than substantially only populations of ependymal and periventricular cells. Again, while secreted polypeptides are also envisioned, the methods are particularly advantageous when non-secreted polypeptides are to be expressed.

In a first aspect, the present invention provides a method of delivering a therapeutic polypeptide to a target CNS cell, more preferably a target neuron or population of neurons, comprising administering a rAAV vector comprising a nucleic acid sequence encoding a therapeutic polypeptide to a neuron or population of neurons synaptically connected to said target CNS cell. The site of administration is thus distant rather than local to a target CNS cell. At least one synapse will separate the target CNS cell from cells at the local site of administration of the rAAV virions. Because the rAAV vector is capable of being transported across a synapse of interconnected neurons, the method involves allowing the vector to transduce synaptically interconnected CNS cells even at significant physical distances, e.g. from the order of micrometers to a meter in the case of motor neurons of great length. In another aspect, transport is effected such that target CNS cells separated by at least 2, 3, 4, 5 or 10 synapses from cells at the site of injection are transduced with the rAAV vector. Transport of the rAAV vectors according to the invention is preferably anterograde transport.

The invention thus preferably involves selecting a target population of neurons to which the therapeutic polypeptide is to be selectively delivered and/or expressed, or to which the rAAV vector is to be selectively transported. Target populations of neurons may be selected according to any suitable means. In general, target populations of neurons for a particular disorder can be found in the literature, based on neuropathological and biochemical studies. Several examples of neuron populations that can be selected are described further below.

Neurons or cell populations of the CNS that are synaptically connected can generally be identified or selected according to any suitable means. Many examples and diagrams of populations of neurons from a first region of the CNS which project to populations of neurons of another region of the CNS by synaptically connected neurons can be found in the literature, for example in N. Marieb (ed.), Human Anatomy and Physiology, 5th ed., Benjamin-Cummings Publishing Company, 2000; Heimer, (ed), The Human Brain and Spinal Cord, 2nd ed., Springer-Verlag; M. L. Barr and J. A. Kiernan, (eds) The Human Nervous System, An Anatomical Viewpoint, 6th ed., J. B. Lippincott, 1993; Burt, A. (ed), Textbook of Neuroanatomy 1st ed., W. B. Saunders, 1993; Kandel E. R., J. H. Schwartz and T. M. Jessel (eds), Principles of Neural Science, 3rd ed., Elsevier, 1991; D. E. Haines (ed), Neuroanatomy: An Atlas of Structures, Sections and Systems, 4th ed., Urban and Schwarzenberg, 1995.

Synaptically connected neurons can also be identified by conducting studies tracing neuronal projections. Synaptic connections can be traced in cultures of neurons in vitro, by administering a rAAV or other suitable (e.g. HSV-1) vector to a non-human test animal, and tracing connections upon sacrificing the animal, or more preferably using a non-human animal model allowing synaptic connections to be visualized and traced in vivo.

In one aspect, the invention also provides a method for identifying synaptically connected populations of neurons comprising administering rAAV virions to a first population of cells in an animal model (e.g. zebrafish), and identifying a second population of cells which are transduced by said rAAV virions. Said animal model may be an in vitro model where the animal is sacrificed (e.g. rodent) or an in vivo model (e.g. zebrafish). Preferably the transduction of several populations of neurons is traced. The rAAV vector may contain a nucleic acid encoding a polypeptide which can be detected with an antibody, or any other detectable polypeptide, such as a fluorescent protein, or an rAAV may comprise a labeled tracer, allowing a defined activity to be followed or detected in vivo. Preferably, the labeled tracer is one that can be viewed in a whole animal, for example, by positron emission tomograph (PET) scanning or other CNS imaging techniques. Suitable labels include, but are not limited to radioisotopes, fluorochromes, chemiluminescent compounds, dyes, and proteins, including enzymes.

Method for the detection of proliferation of rAAV administered to a population of cells of the CNS according to the invention are well known in the art. Detection methods can be used for example to visualize expression of a polypeptide and/or transduction of cells in culture, or of cells in test animals, preferably animal models of disease. In one embodiment, antibodies or in situ hybridization methods may be used to analyze expression of a transgene. In other examples, a fluorescent viral vector can be used to study proliferation and transduction of a vector. Several applications of currently available fluorescence-based detection methods are reviewed in Bartlett et al., Nature Medicine, 4(5):635-637, 1998.

It will also be appreciated that methods of assessing the efficacy of a treatment are well known in the art. Preferably, assessing the efficacy of a treatment comprises detecting or monitoring the amelioration of a symptom in a subject according to standard methods. For example, progression of Alzheimer's disease can be assessed by conducting neuropsychological test on a patient, optionally also monitoring with the use of functional MRI methods. Assessment for motor neuron disease can be carried out using conduction tests, including monitoring response time following a stimulus or muscle force tests.

(a) Alzheimer's Disease (AD)

Alzheimer disease (AD), the major cause (70%) of dementia in adult is a progressive neurodegenerative disorder that occurs in 5% of the population over 65 years of age. It is clinically characterized by a global decline in memory and other cognitive functions that leaves end-stage patients bedriddden, incontinent and dependent on custodial care. Death occurs on average nine years after the diagnosis. The major risk for AD is increasing age and in the USA alone, there are currently over four millions patients with AD.

The major neuropathological changes in the brain of AD patients are neuronal death, particularly in regions related to memory and cognition and the presence of abnormal intra- and extra-cellular proteinaceous filaments. Intracellularly, bundles of paired helical filaments (PHF), composed largely of phosphorylated tau protein and referred to as neurofibrillary tangles, accumulate in large number in dying neurons. Extracellularly, insoluble aggregates of proteinaceous debris, termed amyloid, appear in the form of senile or neuritic plaques and cerebrovascular amyloid deposits. The frequency and distribution of neurofibrillary tangles and neuritic plaques appear to correlate well with extent of cognitive impairment, synaptic loss and neurotransmitter (in particular acetylcholine) depletion. The amyloid deposits consist of aggregates of amyloid β-peptide (M) isoforms. These are 39-42 residue peptides that are proteolytically derived from the large amyloid precursor (APP) by two proteases, β-secretase and γ-secretase, and secreted by all cells. Cells secrete more Aβ40 than Aβ42 isoform that is less soluble and forms the major component of amyloid plaques. The fact that mutations in the APP gene are associated with familial AD is a strong indication of the importance of amyloid in the pathogenesis of the disease. In addition to APP, three other genes have been linked with AD: apolipoprotein E (apoE) on chromosome 19, presenilin (PSI) on chromosome 14 and presenilin 2 (PS2) on chromosome 1. Mutations in PS1 and PS2 and polymorphim in apolipoprotein E gene (ε4 allele) induce an increase in the production or amyloidogenicity of Aβ and are implicated in the formation of amyloid plaques. In turn, diffuse Aβ42 plaques leads to microglial activation, cytokine release, astrocytosis and acute-phase protein release. Progressive neuritic injury occurs within amyloid plaques in a second stage with disruption of neuronal metabolic homeostasis and oxidative injury. At the same time, activities in kinase/phosphatases enzymes are modified leading to hyperphosphorylation of tau protein and the formation of neurofibrillary tangles. Animal models of AD include transgenic mice overexpressing the human APP gene and mutant PSI mice that accumulate senile plaques, present abnormal behavior but without the formation of neurofibrillary tangles. Rats with fimbria-fornix lesions and ages rats develop also atrophy of forebrain cholinergic system associated with cognitive impairments in learning and memory.

Most therapeutic strategies actually in development, such as cholinesterase inhibitors, muscarinic, glutamatergic or serotonergic agonists, anti-inflammatory and anti-oxidant drugs are directed toward palliating existing cognitive symptoms or retarding the disease course. However, preferably, methods of treatment would aim to prevent amyloid deposition. In this aspect of the invention, gene therapy using the methods disclosed herein could be useful in strategies for reducing the Aβ production as follows:

1) Modifying APP processing by:

a) stimulation of a secretase cleavage to divert substrate from the Aβ-forming β-secretase pathway. Various studies suggest that activation of neurotransmitter receptors coupled to protein kinase C signaling cascade stimulate the α-secretase pathway at the expense of the β-secretase pathway; or

b) inhibition of β- and γ-secretase pathways. However, since of β- and γ-secretases are present in many different CNS cells, it is reasonable to assume that they have substrates in addition to APP. Complete inhibition of these secretases in all CNS cells might result in toxicity. It is therefore important to target specifically the inhibition of these secretases in specific neuronal populations. PS1 and PS2 might have γ-secretase activity or at least interact with the γ-secretase. A transmembrane protease termed β site APP cleaving enzyme (BACE) with all known properties of β-secretase has recently been identified.

2) Using gene transfer to target antisense oligonucleotides or genetically engineered ribozymes to reduce APP gene expression. Again in this case, global lowering of APP might produce undesirable side effects. For example, homozygous APP-deficient mice develop various abnormalities, including decreased body weight, reduced locomotor activity, spontaneous seizures impaired behavioral performance and premature death. Thus, targeting of antisense oligonucleotides or genetically engineered ribozymes in specific neuronal population is likely to be necessary.

Gene therapy for AD might involve the transfer of genes that enhance survival and functions of neurons. NGF is a promising gene given it protects cholinergic neurons from axotomy-induced cell death in fimbria-fornix lesion models, reverses age-associated atrophy of cholinergic cell bodies and improves spatial navigation, memory and learning in mice. Other candidate “therapeutic” genes include those that code for protein that inhibit tau protein hyperphosphorylation that leads to the formation of neurofibrillary tangles.

Methods of Administration for the Treatment of AD

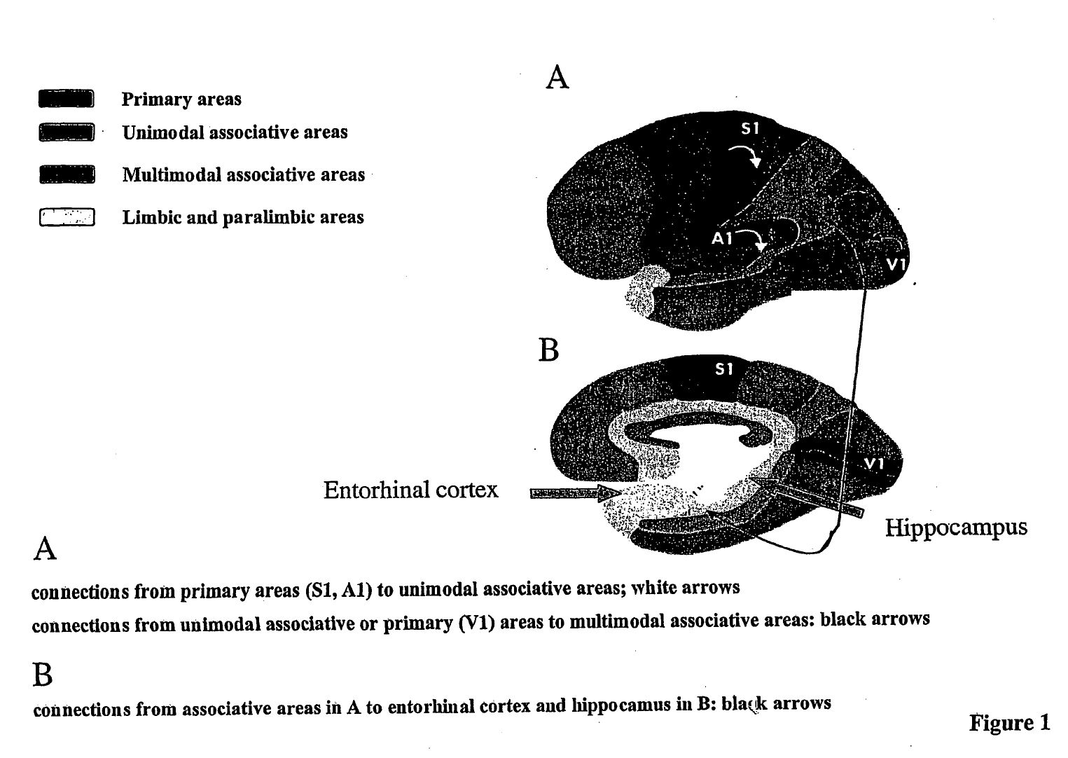

At an end-stage, AD involves widespread populations of neurons within the brain. Due to the limited capacity to express therapeutic genes after cerebral injection, gene therapy of AD is considered as a remote prospect. Although amyloid deposition is the first abnormality to be observed, it is always associated to the formation of neurofibrillary tangles. Neuropathological and biochemical studies show that the progression neuronal lesions follow particular neuronal connections. At the initial and asymptomatic stage, when neuronal loss is present in the hippocampus, the entorhinal cortex (which is localized at the internal surface of temporal lobe) is always involved. Conversely, when the entorhinal cortex is involved, the hippocampus may be spared. Thus, the involvement of entorhinal cortex follows that of hippocampus. The entorhinal cortex serves as an interface between the hippocampus and the different associative areas of the brain cortex (FIG. 1). Next, the superior temporal lobe is involved followed by the inferior and medium temporal lobe.

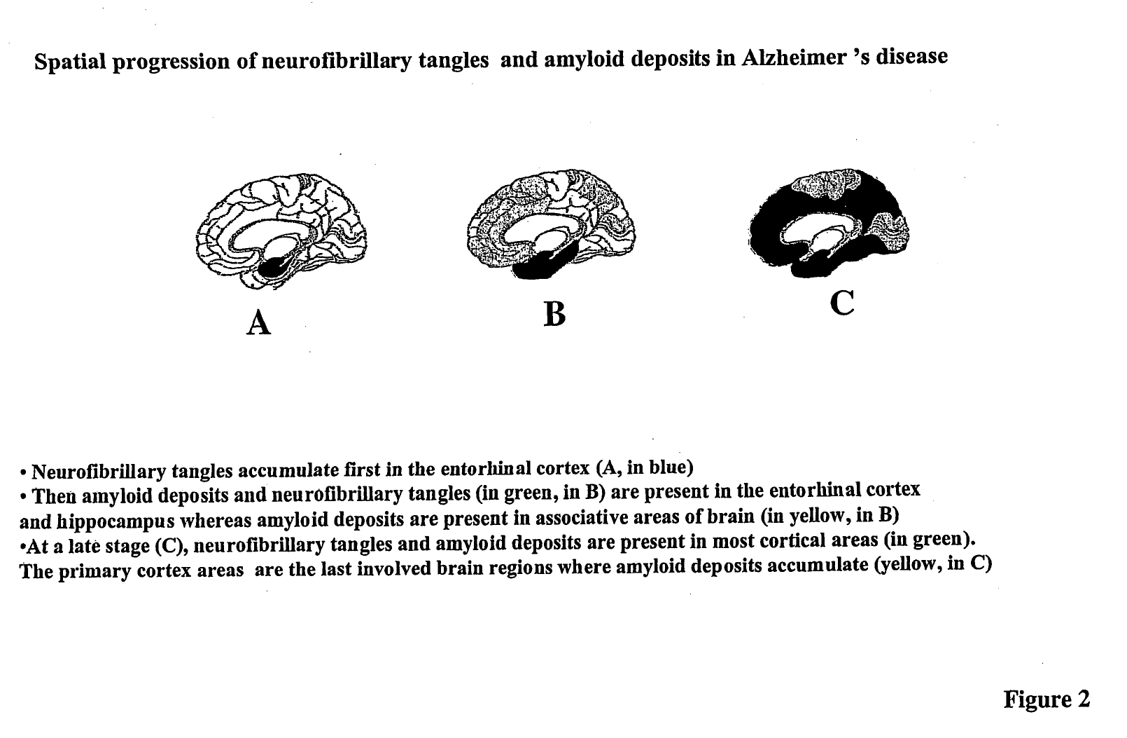

At the initial stage, the spatial distribution of amyloid debris is already diffuse whereas brain neurofibrillary tangles are restricted to entorhinal cortex (FIG. 2). The neurofibrillary tangles extend then in hippocampus, associative cortex areas and only at a late stage in primary (sensory and motor) areas. This progression suggests that neuronal connections play a major role in the progression of the disease. These projections can be anterograde (from primary cortex areas to hippocampus) or retrograde (from hippocampus to primary cortex areas). Neurofibrillary tangles are present in layers II, III and V whereas neuritic plaques are present in layers II and III of the cerebral cortex. The spatial distribution of neurofibrillary tangles and neuritic plaques suggests that anterograde connections play a major role. Another argument for the role of neuronal connection is the fact that neuronal degeneration is not always associated with the nearby presence of amyloid plaques. This can be explained by the fact that neurons initially affected by the degenerative process project at distance to specific other neurons and that vulnerability of specific neuronal population are directly related to the connections that link them.

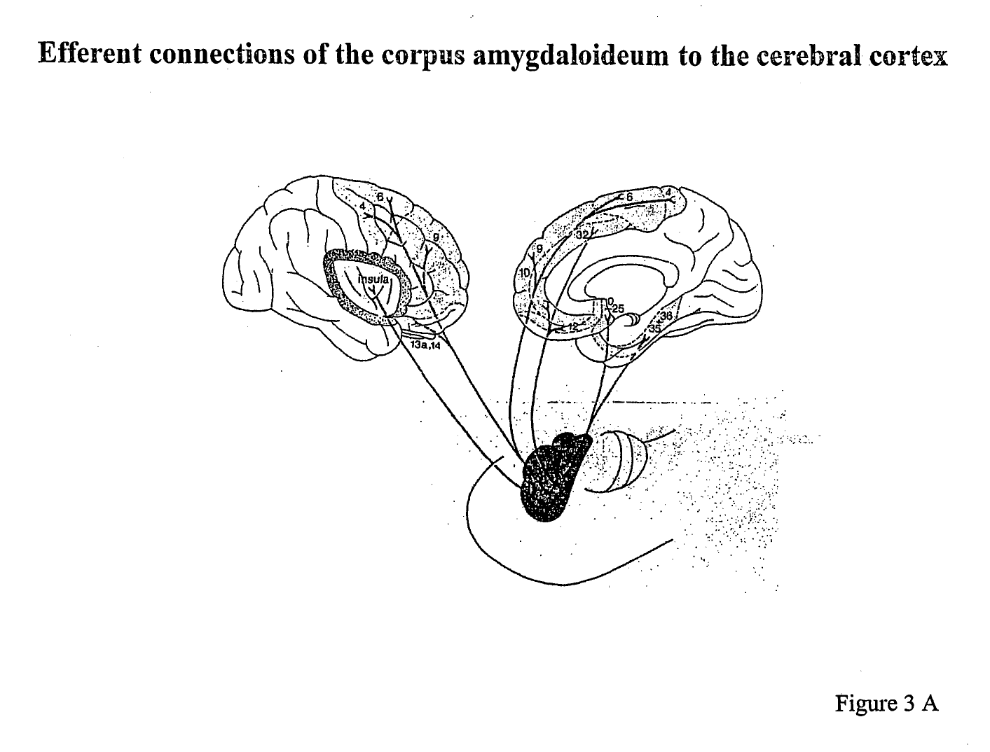

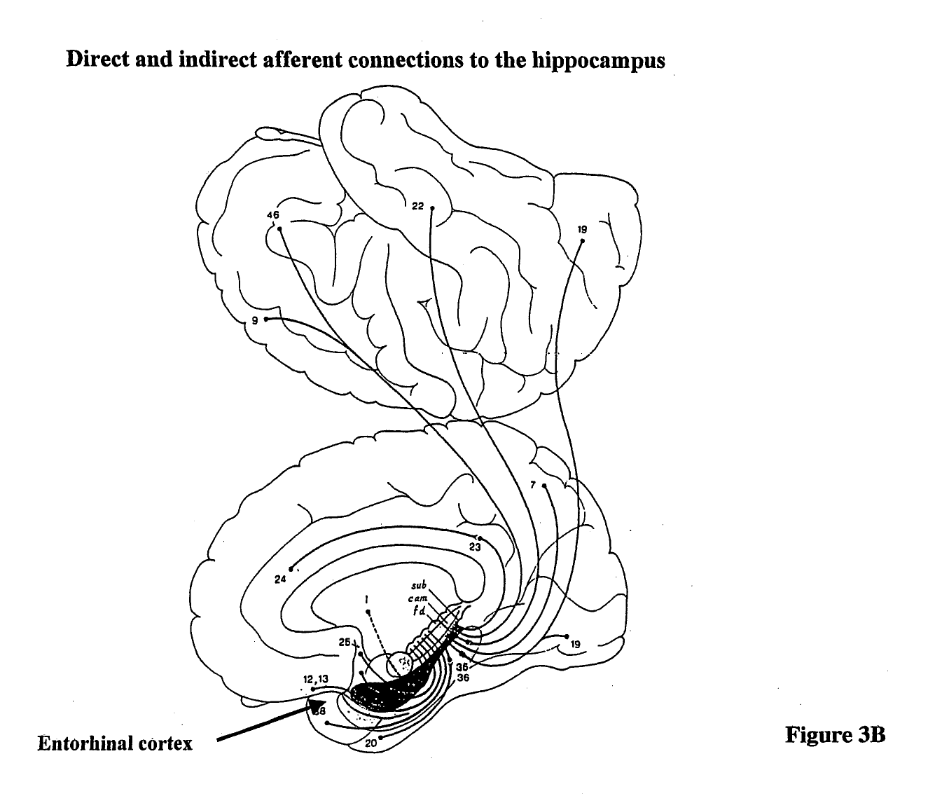

As a result of the discovery that anterograde transport of AAV particles is followed by transynaptic transport, allowing expression of therapeutic genes in remote connected neurons, the present invention provides for the targetting of therapeutic genes in the corpus amygdaloideum that send anterograde projections to various associative brain areas (FIG. 3A). Injection of gene therapy vectors in the entorhinal cortex would also to target recombinant particles in the hippocampus that in turn send projections in associative areas (7, 9, 19, 22 and 46) (FIG. 3B).

Motor Neuron Diseases (MNDs).

Motor neuron diseases (MNDs) involve lesions in one or both components of a two-neuron pathway. These disorders are severely debilitating and often fatal. One potential treatment for MNDs is gene therapy and to discuss this approach, the two-neuron pathway under assault that involves these diseases must be taken into account. The cell bodies of upper motor neurons, located in layer V of the primary motor cortex in the cerebral cortex project long axons to the spinal cord and brainstem, where they make synaptic contact with lower motor neurons. The lower motor neurons in turn project axons out through cranial and spinal nerves to synapses on muscle fibers of the head and body, respectively. Motor activity occurs when upper motor neurons excite lower motor neurons, which in turn stimulate muscular contraction. Lower motor neurons can extend a great distance, in some cases 1 meter or more from the cell body in the spinal cord.

(a) Amyotrophic Lateral Sclerosis

Amyotrophic lateral sclerosis (ALS) is characterized by neurodegeneration of both lower and upper motor neurons. Onset is in the fourth to fifth decade leading to death from neuromuscular respiratory failure within 2-5 years. The prevalence of ALS is estimated 1/20.0000 and incidence of familial cases occurs in 10%. In these affected individuals, onset of ALS is a decade earlier and neurons present Lewy body inclusions that are not present in sporadic cases. 90% of familial ALS cases are autosomal dominant and 10% are autosomal recessive. Mutations in superoxide dismutase 1 (SOD1) are responsible for most of autosomal dominant and some autosomal recessive forms of ALS. The frequency of SOD1 mutations in sporadic ALS range from 2 to 7%. Transgenic mice overexpressing mutant human SOD1 is a good mouse model of ALS. These mice develop degeneration of spinal cord neurons similar to human patients with ALS, including the presence of phosphorylated neurofilaments and Lewy body-like inclusions. Mutant SOD1 mice have a gain of function, with survival inversely related to SOD1 activity. The different gene therapeutic approaches that are envisaged in ALS aim at: transferring genes that can protect against SOD1 mutant toxicity. The mutant SOD1 produces peroxynitrite or nitrosamine peroxide that leads to mitochondrial dysfunction, increased cytosolic calcium and subsequent neuronal apoptosis. The calcium-buffering protein calbindin could be in particular protective. Transferring proto-oncogene bcl-2, interleukin 1-converting enzyme or other genes that can counteract SOD1 mutant toxicity by inhibiting apoptotic cell death. For example, Azzouz et al., Human Mol. Genetics, 9(5):803-811, 2000, suggest increased motor neuron survival and improved neuromuscular function in transgenic ALS mice after intraspinal injection of a rAAV vector comprising a nucleic acid encoding Bcl-2. Transferring genes that encodes trophic factors were shown to slow down the progression of motor neuron degeneration in transgenic SOD1, motor neuropathy (pmn) mutant mice or after axotomy-induced degeneration in animal models. BDNF, GDNF, NT-3 and IGF-1 trophic factors are the best candidates. Expressing genes that could decrease glutamate toxicity that is observed in patients with ALS and various animal models of MNDs.

(b) Spinal Muscular Atrophy (SMA)

Spinal muscular atrophy (SMA) is another genetic (autosomal recessive) MND whose incidence is 1/10,000 live births. SMA affects mainly infants before 2 years of age and is characterized by progressive degeneration of spinal motor neurons. SMA is one of the most common inherited causes of childhood mortality. Patients with SMA have mutations (often deletions) in survival motor neuron (SMN) and neuronal apoptosis inhibitory protein (NAIP). Humans have 2 copies of SMN genes (SMN1 and SMN2); only mutation of SMN1 is causative of SMA. SMN1 protein, which has functions in splicing of several genes, is reduced by 100-fold in the spinal cord of SMA patients. In contrast to human, mouse has only one copy of SMN gene and complete inactivation of this gene leads to embryonic lethality. SMN -/- mice transgenic for human SMN2 gene and conditional SMN -/- mice develop degeneration of spinal motor neurons after birth that resemble to SMA. The different therapeutic approaches that are envisaged in SMA aim at: overexpressing SMN2 gene expression that could result in less neurodegeneration. Transferring NAIP gene that inhibits apoptosis from glutamate exposure and as in ALS transferring in motor neurons trophic factor genes and/or genes that could decrease glutamate toxicity.

(c) Kennedy's Disease (Bulbospinal Atrophy)

Kennedy's disease (bulbospinal atrophy) is a rare X-linked MND that begins in the fifth to sixth decade. Kennedy's disease is caused by an expansion of CAG trinucleotide repeat in the androgen receptor gene, rendering the truncated gene product unstable. The result is reduction in gene levels whose expression is regulated by the androgen receptor. The different therapeutic approaches that are envisaged in Kennedy's disease aim at: gene transfer of chaperone proteins to motor neurons in brainstem and spinal cord. Studies in vitro have shown that incomplete loss of androgen receptor product can be overcome by overexpression of a heat shock chaperone protein and as in ALS transferring in motor neurons trophic factor genes and/or genes that could decrease glutamate toxicity.

(d) Hereditary Spastic Hemiplegia (Paraplegia)

Another group of MNDs are hereditary spastic hemiplegia (paraplegia). These MND involve upper motor neurons in cerebral cortex and their corticospinal projections to spinal cord that innervate the lower limbs. Penetrance (from mild impairment to severe paralysis) and onset can be variable. These genetic MNDs are inherited in autosomal-dominant, recessive and X-linked fashions. Several different genes have been identified including the [symbol] spastin [symbol] gene whose mutations account for 40-50% of autosomal dominant spastic hemiplegia. Spastin is a nuclear-coded mitochondrial protein that is part of the AAA super family (ATPase Associated with diverse cellular Activities). Mutations in paraplegin are responsible for recessive spastic hemiplegia and paraplegin is another nuclear-coded mitochondrial ATPase protein with probable proteolytic and chaperone functions at the inner mitochondrial membrane. Animals models whose spastin or paraplegin genes have been inactivated are currently being prepared and analyzed. The motor neuron abnormalities observed in these animal models will lead to propose specific therapeutic approaches aimed at slow down or arrest the degeneration of motor neurons in cerebral cortex.

Methods of Administration for the Treatment of MND

Therapy by direct gene transfer in MND may require that vectors transduce up to 1 million corticospinal upper neurons and 100,000 to 200,000 lower motor neurons having widespread distribution along the spinal cord. One method is to directly inject rAAV into the brain and the spinal cord but this will require vector distribution after injection such that transduction to large number of cells can occur. Another method is to take advantage of retrograde transport. Lower motor neurons could be transduced after injection into muscle or peripheral nerves provided viral particles are taken up by neuromuscular terminals and axons and transported back to neuronal cell bodies. Adenovirus, recombinant HSV (herpes simplex type 1) and nonviral plasmid liposome vectors can transduce lower motor neurons via retrograde transport.

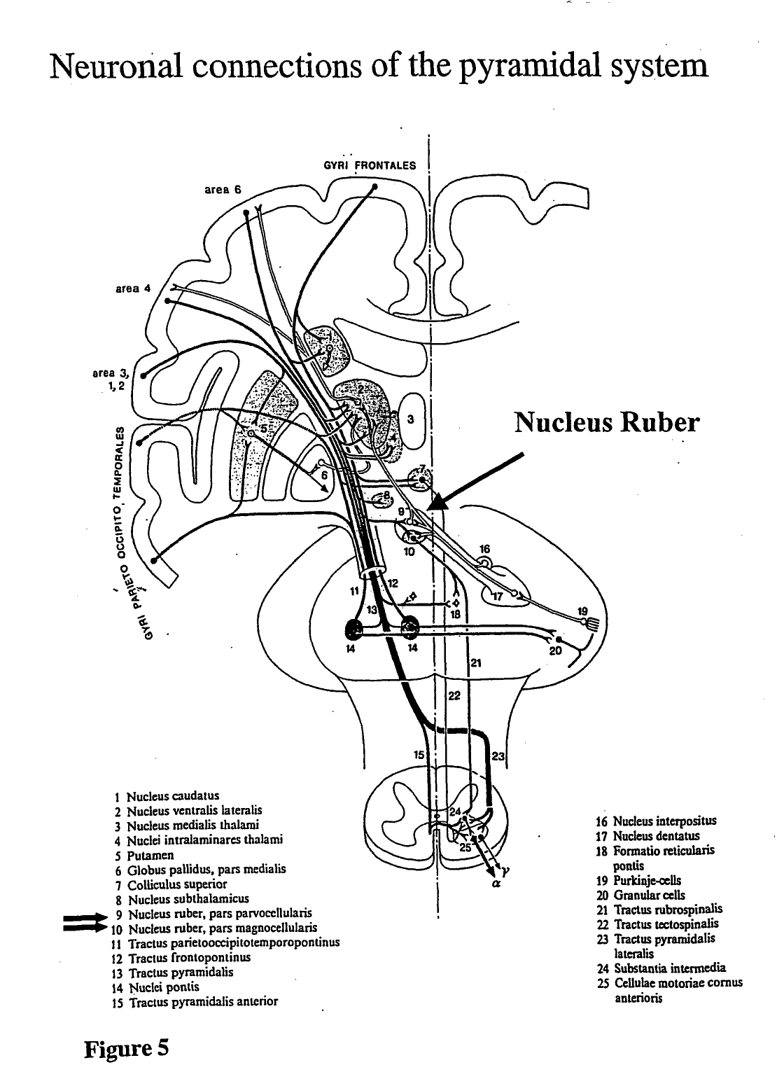

However, in view of the discovery that anterograde transport of AAV particles is followed by transynaptic transport, the present invention provides improved means for achieving expression of therapeutic genes in remote connected neurons. According to the present invention, therapeutic genes are selectively targetted to limited and defined brain structures like the ruber nucleus that send projections to scattered motor neurons in the spinal cord (FIG. 5). Descending fibers of the rubrobulbar and rubrospinal tracts from the contralateral red nucleus terminate on interneurons in the lateral reticular formation and the dorsolateral intermediate zones of the spinal cord and directly on spinal cord motor neurons. Lesions of the rubrospinal tract result in motor deficits in the execution of independent movements of the limbs, especially of their distal parts.

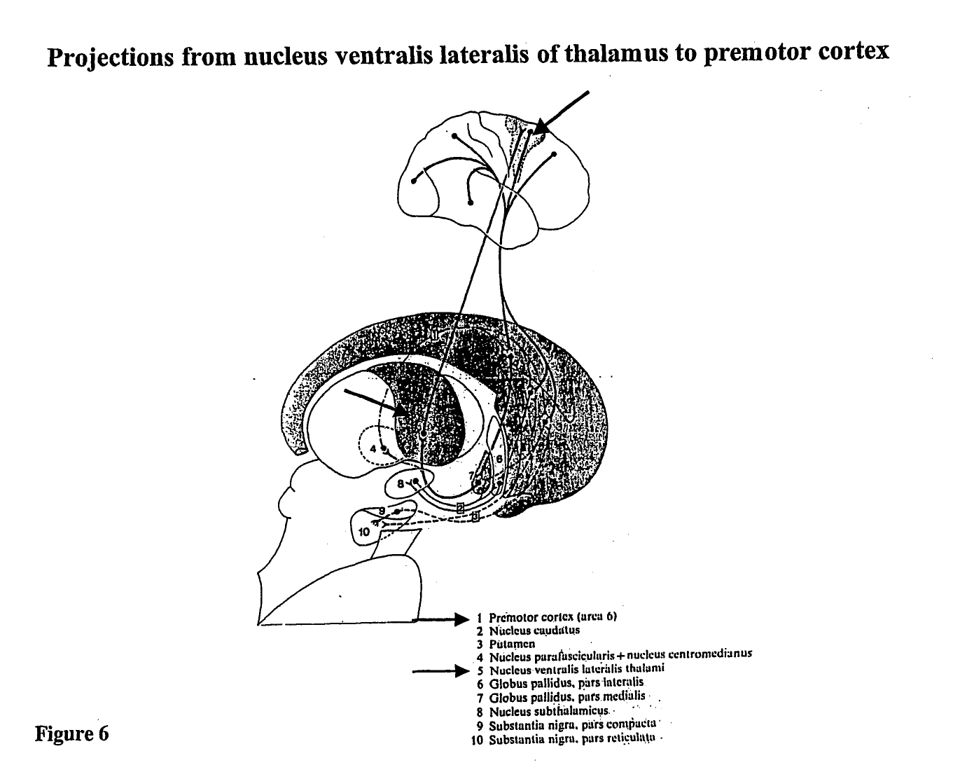

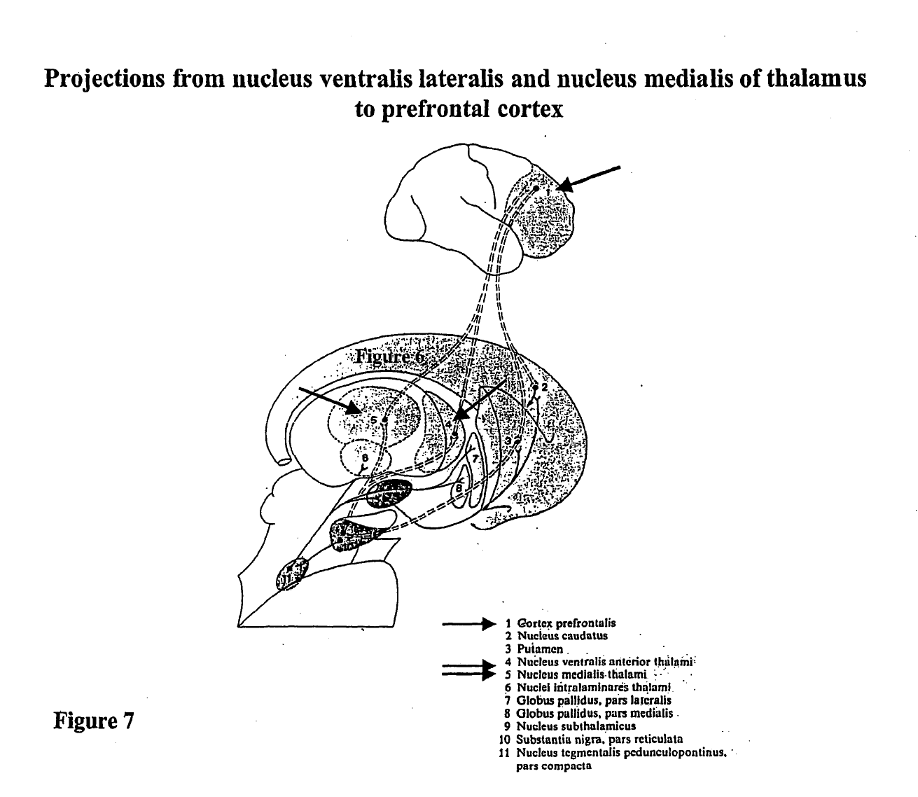

In another preferred embodiments the invention also comprises transducing widespread populations of upper motor neurons located in the premotor cortex (area 6) and cortex prefrontalis after injection of therapeutic gene vectors in the ventralis lateralis and/or anterior nuclei of thalamus (FIGS. 6 and 7). One major advantage of such approaches would be that the transduction of a relatively limited number of neurons in specific brain regions would be sufficient to transduce large number of motor neurons having widespread localization in motor cerebral cortex or along the spinal cord.

Human Adrenoleukodystrophy (ALD)

X-linked adrenoleukodystrophy (ALD) is a monogenic peroxisomal disorder characterized by diffuse demyelination within the CNS. ALDP, an integral component of the peroxisomal membranes, belongs to the family of ABC transporters and is involved in the degradation of very-long-chain fatty acids (Aubourg (2000), supra). A gene and protein responsible for adrenoleukodystrophy was identified and cloned, further described in U.S. Pat. No. 6,013,769.

As described in detail in the Examples below, adrenoleukodystrophy (ALD) can be treated by co-administering a rAAV vector expressing HALD into the CNS thereby restoring hALD function.

An AAV vector was used to deliver the human ALD gene to the brain of adult ALD mice through the injection of AAV vector in lumbar spinal cord. This induced long standing expression of ALDP expression in neurons localized in thalamus and colliculus, indicating that AAV particles bearing therapeutic genes undergo anterograde transport and neuron to neuron passage over long distances in the central nervous system. Results were confirmed in studies using injections in the brain of adult and newborn ALD mice.

All the method for delivering recombinant AAV virions to a subject, according to the present invention, methods which can be considered as method of treatment of an animal or a human body, could be converted as claims of use of rAAV virions carrying a transgene in the preparation of a medicament for the treatment of a disease in a subject wherein the characteristics of the claims methods in claims 1 to 77 can be included without limitations.

So, in another aspect of the present invention, the invention also comprises the use of rAAV virions carrying a transgene in the preparation of a medicament for the treatment of a disease in a subject,

wherein a first population and a second population of synaptically connected neurons are selected and a therapeutic polypeptide is to be expressed in said second population of neurons;

and a medicament comprising recombinant adeno-associated virus (rAAV) virions is delivered to said first population of neurons of the subject, wherein said virions comprise a nucleic acid sequence that is expressible in transduced cells to provide a therapeutic effect in the subject, and wherein said rAAV virions are capable of transducing synaptically connected neurons.

In a preferred embodiment, the present invention comprises the use according to the present invention, wherein said rAAV virions are capable of being transported across at least one synapse between said first and said second populations of connected neurons.

In another preferred embodiment, the present invention comprises the use according to the present invention, wherein said first and said second populations of neurons are separated by at least one, two or three synapses.

In another preferred embodiment, the present invention comprises the use according to the present invention, wherein said rAAV virions transduce cells consisting essentially of neurons synaptically connected to one another.

In another preferred embodiment, the present invention comprises the use according to the present invention, wherein said second populations of neurons is a population of motor neurons.

In another preferred embodiment, the present invention comprises the use according to the present invention, wherein said rAAV is a AAV-2, AAV-4 or AAV5 subtype.

In another preferred embodiment, the present invention comprises the use according to the present invention, wherein the administration comprises direct intracerebral administration, intrathecal administration or stereotactic microinjection.

In another preferred embodiment, the present invention comprises the use according to the present invention, wherein the polypeptide is a non-secreted or a secreted polypeptide.

In another preferred embodiment, the present invention comprises the use according to the present invention, wherein the nucleic acid sequence encodes a polypeptide capable of preventing or decreasing the rate of degeneration of a neuron.

In another aspect, the present invention comprises the use according to the present invention, wherein said disease is a neurodegenerative disease, preferably Alzheimer's disease.

In a preferred embodiment, the present invention comprises the use according to the present invention, wherein said preparation is delivered to the corpus amygdaloideum or to the entorhinal cortex of the subject.

In another preferred embodiment, the present invention comprises the use according to the present invention, wherein the therapeutic polypeptide is a polypeptide capable of inhibiting or reducing the formation of Aβ production, capable of modifying APP processing, capable of stimulating α-secretase cleavage activity, capable of inhibiting the β-secretase pathway, capable of inhibiting the γ-secretase pathway, capable of inhibiting tau protein hyperphosphorylation.

In another preferred embodiment, the present invention comprises the use according to the present invention, wherein said rAAV virions comprise a nucleic acid sequence encoding an antisense nucleic acid or a catalytic RNA capable of reducing APP gene expression.

In another preferred embodiment, the present invention comprises the use according to the present invention, wherein said rAAV virions, said second populations of neurons, said rAAV, said first and said second populations of neurons, the administration or the polypeptide are as described above.

In another aspect, the present invention comprises the use according to the present invention, wherein said disease is a motor neuron disease, preferably amyotrophic lateral sclerosis (ALS).

In a preferred embodiment, the present invention comprises the use according to the present invention, wherein rAAV virions are delivered to the ruber nucleus, to the ventralis lateralis or to the anterior nuclei of the thalamus.

In another preferred embodiment, the present invention comprises the use according to the present invention, wherein said therapeutic polypeptide is superoxide dismutase 1 (SOD1), is a polypeptide capable of inhibiting apoptotic cell death or a trophic factor.

In another aspect, the present invention comprises the use according to the present invention, wherein said motor neuron disease is SMA.

In a preferred embodiment, the present invention comprises the use according to the present invention, wherein said therapeutic polypeptide is SMN2, a trophic factor or a polypeptide capable of decreasing glutamate toxicity.

In another aspect, the present invention comprises the use according to the present invention, wherein said motor neuron disease is Kennedy's disease (bulbospinal atrophy).

In a preferred embodiment, the present invention comprises the use according to the present invention, wherein said therapeutic polypeptide is a chaperone polypeptide, a polypeptide capable of increasing chaperone polypeptide expression, a trophic factor or a polypeptide capable of decreasing glutamate toxicity.

In another aspect, the present invention comprises the use according to the present invention, wherein said motor neuron disease is paraplegia.

In a preferred embodiment, the present invention comprises the use according to the present invention, wherein said rAAV virions, said first and said second populations of neurons, said second populations of neurons, said rAAV, the administration or the polypeptide are as described above.

In a more preferred embodiment, the present invention comprises the use according to the present invention, wherein the subject is a human.

In a preferred embodiment, the present invention comprises the use according to the present invention, further comprising administering to the subject at least one additional therapeutic compound.

Having generally described this invention, a further understanding can be obtained by reference to certain specific examples which are provided herein for purposes of illustration only, and are not intended to be limiting unless otherwise specified.

### EXAMPLES

### Example 1

### Construction and Production of AAV-hALD

A recombinant AAV vector (PGK-hALD-AAV) was engineered to contain the human ALD cDNA (hALD) (Mosser et al., Nature, 361:726-730, 1993) under the control of the mouse phosphoglycerate kinase (PGK) promoter. The PGK-hALD cassette was obtained from the M48-ALD vector (Cartier et al., Proc. Natl. Acad. Sci. USA, 92:1674-8, 1995) and inserted upstream the SV40 polyadenylation site between the two ITR of pSUB201 deleted for rep and cap sequences (Samulski et al., J. Virol., 63:3822-8, 1989). AAV vector stocks were prepared and titered as previously described (Salvetti et al, Hum Gene Ther., 9:695-706, 1998). The vector preparation contained 1.7*10⁹ infectious particles/ml.

The entire cassette was flanked by AAV inverted terminal repeats (ITRs) that are required for gene expression, replication, and packaging into viral particles. Recombinant AAV virions were produced in human 293 cells (readily available through, e.g., the American Type Culture Collection under Accession Number ATCC CRL1573) as follows. The 293 cell line was cultured in complete DMEM (Biowhittaker) containing 4.5 g/liter glucose, 10% heat inactivated fetal calf serum (FCS; Hyclone), and 2 mM glutamine. Subconfluent 293 cells were co-transfected by calcium phosphate precipitation (see, e.g., Sambrook, et al.) with the AAV-tk expression cassette flanked by ITRs and helper plasmids derived from both AAV (pw 1909, containing the AAV rep and cap genes) and adenovirus (pLadenol, containing E2a, E4, and adenoviral VA, and VA, I RNA genes). After 6 hours, the media was changed to DMEM without serum and incubation was continued at 37° C. in 5% C02 for 72 hours. Pelleted cells were lysed in Tris buffer (100 mM Tris/I 50 mM NaCl, pH 8-0) by three cycles of freeze/thaw, and lysate was clarified of cell debris by centrifugation at 10,000 g for 15 in. To pellet non-viral proteins, the clarified lysate was centrifuged at 10,000 g for 15 min after adding CaCl2 to a final concentration of mM and incubated for 1 h at 0° C. Polyethylene glycol 8000 (PEG) was added to the resulting supernatant (final concentration=8%); this solution was incubated for 3 h at 0° C. and centrifuged at 3000×g for 30 minutes. The vector containing pellet was solubilized in 50 mM Hepes Na/150 mM NaC1/25 mM EDTA, pH 8.0, and centrifuged at 10,000×g for 15 minutes to pellet and remove insoluble material. Cesium chloride isopycnic gradient centrifugation was performed and AAV-tk was recovered from the resulting gradient by isolating the fractions with in average density of 1.38 g/ml. PEG was again used to concentrate vector, which was then resuspended in 25 mM Hepes Na/I 50 mM NaCl, pH 7.4 and centrifuged as described to remove insoluble material. The stock was treated with DNAse and vector titer was determined by quantitative dot-blot hybridization.

### Example 2

### In Vivo Delivery of AAV-HALD

ALD deficient mice were originally obtained from Dr. K. Smith (Baltimore, Md., USA) (Lu et al., Proc. Natl. Acad. Sci., U.S.A., 94:9366-9371, 1997).

ALD newborn mice were anesthetized on ice. A small burr hole was drilled in the skull with a 26-G needle and a glass micropipette was introduced into the subventricular zone of the left lateral ventricle (AB). Adult mice were anesthetized by intraperitoneal administration of a mixture of ketamine (Panpharma, Luitre-Fougeres, France)/xylazine (Sigma, St Quentin-Fallavier, France) (0.1/0.01 mg/g body weight). The anesthetized mice were mounted onto a stereotaxic frame (David Kopf Instruments, Tujunga, Calif.). The skull was exposed and holes were drilled bilaterally for infusion in the corpus callosum (1.1 mm rostral and 1.3 mm lateral to bregma, depth 2 mm) and pons (4.6 mm caudal and 1 mm lateral to bregma, depth 4.25 mm) according to the atlas of Franklin and Paxinos (AC). PGK-hALD-AAV vector was delivered at each injection site with an ultrapump (World Precision Instruments, Sarasota, Fla., USA) at a rate of 0.4 MI/min for 5 min. The micropipette was left in place for an additional 5 min to allow diffusion from the injection site and then slowly withdrawn. The scalp was closed and animals were returned to recovery cages.

### Example 3

### Analysis of hALD Expression

After deeply anesthetized animals were sacrificed, brain, cerebellum and spinal cord were removed, frozen into isopentane and stored at −70° C. until analysis. Serial sections (4 mm thick) of brain, cerebellum and spinal cord were cut at −17° C. using a cryostat, fixed in 4% formaldehyde for 15 min and permeabilized in PBS-Triton X-100, 0.1%. Sections were washed three times in PBS for 5 min and incubated with the first antibody at 37° C. or at room temperature for 30 minutes. Immunohistochemical analysis of human ALDP was performed using a rabbit polyclonal or mouse monoclonal anti-hALDP antibody that does not crossreact with mouse ALDP (Franklin K B J; Paxinos G., 1997. The mouse brain: in stereotaxic coordinates. San Diego: Academic Press; and Fouquet et al, Neurobiol. Dis., 3:271-285, 1997). ALDP immunostaining gives a characteristic punctuate pattern that reflects the distribution of peroxisomes in the cell body and processes of CNS cells. Peroxisomes are mostly located around the nucleus, allowing the identification of transduced cells. (Fouquet et al, 1997, supra; and Cartier et al., 1995, supra). Neurons were stained with mouse monoclonal antibody against mouse neuronal nuclei (NeuN) (Chemicon, Temecula, Calif., USA); oligodendrocytes with mouse monoclonal antibody against human 2′-3′-Cychc Nucleotide 3′ Phosphodiesterase (CNP) (Chemicon, Temecula, Calif., USA); astrocytes with guinea-pig anti-human glial fibrillary acid protein, GFAP (Chemicon, Temecula, Calif., USA); and microglia with Ricinus Communis Agglutin (RCA) directly labeled to fluorescein isothiocyanate (FITC) (Sigma, Saint-Louis, Mo.). All secondary antibodies were obtained affinity-pure from Jackson Immunoresearch (Westgrove, Pa., USA) as follows: biotinylated goat anti-rabbit IgG (H+L) followed by Cy3 or FITC streptavidin, FITC-conjugated horse anti-mouse IgG (H+L). All primary antibodies were diluted in PBS-Triton X-100, 0.01% with 500 ug/ml goat IgG or 5% goat serum while all subsequent incubations or washes were done in PBS Triton X-100, 0.01%. Appropriate filters for each fluorochrome and combined filters for fluorescein and Cy3 were used on light microscope equipped with fluorescence (Nikon Optiphot-2).

Mice half brain studies consisted of 570 serial slices 7-8 μm thick. In a preliminary step, we established that the percentage of ALDP positive cells followed a linear diminution from the injection site to the most external site of brain slices. ALDP positive cells were counted on 6 adjacent slices at the site of injection, 6 slices at 3 μm from this site and on 6 slices in a region localized half between these 2 sites. The number of positive ALDP cells decreased proportionally from the injection site (R²=0.94 and 0.95 respectively) to the periphery. The total number of transduced cells on each slice was calculated with an appropriate formula and corrected for the likelihood of counting the stained neuron repeatedly on adjacent slices.

### Example 4

### Gene Therapy of Adrenoleukodystrophy

X-linked adrenoleukodystrophy (ALD) is a monogenic peroxisomal disorder characterized by diffuse demyelination within the CNS. ALDP, an integral component of the peroxisomal membranes, is an intracellular nonsecreted protein from the ATP-binding cassette (ABC) family and belongs to the family of ABC transporters and is involved in the degradation of very-long-chain fatty acids (Dubois-Dalcq M, Feigenbaum V, Aubourg P. The neurobiology of X-linked adrenoleukodystrophy, a demyelinating peroxisomal disorder. Trends Neurosci., 22:4-12, 1999; Aubourg and Dubois-Dalcq, Glia, 29: 186-190, 2000).

Examples 1 to 3 relate to the preparation and administration of an AAV vector used to deliver the human ALD gene to the brain of adult ALD mice through the injection of AAV vector in lumbar spinal cord. Results demonstrate that long standing expression of ALDP was induced in neurons localized in thalamus and colliculus, indicating that AAV particles bearing therapeutic genes can undergo anterograde transport and neuron to neuron passage over long distances in the central nervous system.

This phenomenon using injections in the brain of adult and newborn ALD mice. This possibility was evaluated by following the diffusion of the human adrenoleukodystrophy (ALD) gene in the central nervous system after injection of engineered AAV in the spinal cord, corpus callosum and pons of ALD deficient mice.

Seven weeks after injection of PGK-hALD-AAV in the left posterior horns of lumbar spinal cord (n=2), many ALDP positive cells were present at the site of injection. A search for ALDP positive neurons in the spinal cord and brain was conducted, with specific attention to areas connected with posterior horns of the spinal cord (spino-thalamic tracts). ALDP positive neurons were found in ipsilateral posterior colliculus and ventro-lateral nucleus of thalamus. Because afferent and efferent axons connect the colliculus to the spinal cord, the presence of ALDP positive cells in the colliculus results from either retrograde or anterograde transport of AAV particles followed by transysnaptic transport. However, no thalamic neurons are known to project to spinal cord. The expression of ALDP in thalamus therefore indicates the anterograde transport of AAV particles along the spino-thalamic axonal pathway.

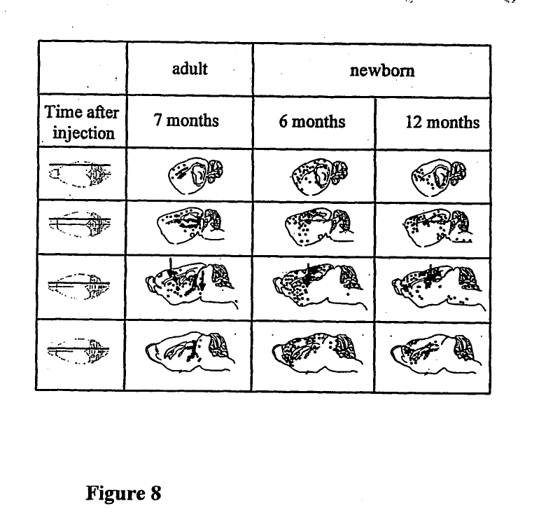

2 μl of PGK-hALD-AAV vector were simultaneously injected stereotactically in the corpus callosum and pons of 7 adult ALD mice. Analysis of ALD protein expression was performed in the injected hemisphere at 5 weeks (n=1), 3 months (n=3) and 7 months (n=2) (FIG. 8). In the corpus callosum injection area, most transduced cells were neurons localized above and below this myelinated structure (FIG. 8). ALDP expression was detectable in axons that lined inside the corpus callosum but not in oligodendrocyte, astrocytes and microglia bodies or processes (not shown). A large population of ALDP positive neurons was observed in the pons around the second site of injection (FIG. 8). Many ALDP positive neurons were found in specific areas connected with both injection sites (FIG. 8): the anterior cerebral cortex, olfactory bulb, striatum, thalamus, optic nuclei, inferior colliculus and even in the cervical spinal cord. ALDP positive neurons located between these remote areas and the injection sites were often lined up and connecting axons could be traced by immunostaining of peroxisomes with ALDP antibody. ALDP expression was comparable in all treated animals and remained stable up to 7 months.

2 μl of PGK-hALD-AAV stock were injected at post-natal day 1 (P1) in the SVZ of 10 ALD newborn mice. The SVZ contains neural precursors that differentiate in olfactory bulb neurons and glial cells after birth in rodents (Alvarez-Buylla et al, 2000, Prog. Brain Res. 127:1-11; and Lim et al., 1997, Proc. Natl. Acad. Sci. U.S.A., 94:14832-14836). ALDP expression was studied in the injected (FIG. 8) and opposite hemispheres of mice at times ranging from 2 weeks to 12 months. Numerous ALDP positive cells were present at the injection site around the ventricle (FIG. 8) but many ALDP positive cells were also present at significant distance from the injection site (FIG. 8), not only in the olfactory bulb, adjacent cerebral cortex and striatum, but also in thalamus, hippocampus and even in the optic nuclei, pons, cerebellum, brain stem and cervical spinal cord. ALDP immunostaining was observed in only few ependymal cells lining the ventricle and in choroid plexus. ALDP expression extended approximately 3 mm laterally and 7 mm in a rostro-caudal direction from the injection site. ALDP positive cells present in the cervical spinal cord were approximately at 18 mm from the injection site. Double labeling with specific markers for neurons, astrocytes, oligodendrocytes and microglia revealed that most ALDP expressing cells were neurons. Analysis of the non-injected hemisphere showed ALDP positive cells in the olfactory bulb, ports, cerebral cortex and cerebellum of all treated mice (not shown). Quantitative analysis of ALD positive neurons in the 10 treated ALD newborn mice showed that, starting with 145±65 ALDP positive cells at the injection site, 55±36 ALDP positive cells were present laterally at 2 mm from this site. The total number of ALDP positive cells per half brain ranged approximately from 19,000 to 78,000. Some individual variation was observed but no significant decrease of ALDP positive cell number was observed up to 12 months.

Since ALD protein is a non-secreted peroxisomal transmembrane protein, ALD can be considered as a good paradigm for the evaluation of gene therapy in this category of CNS disease. Given that AAV type 2 vector has a neuronal tropism (Kaplitt et al., 1994, supra; Mandel, 1997, supra; Alexander et al., Hum. Gene Ther., 1996, 7:841-850; Bartlett et al., Hum. Gene Ther., 1998, 9:1181-1186), this Example provides a demonstration of the extent of neuronal ALD protein expression after direct intracerebral injections.

Thus, the presence of neurons expressing ALD protein in the thalamus after lumbar spinal cord injection showed that AAV particles were anterogradely transported from spinothalamic nerve terminals in the spinal cord to thalamic neurons. Two simultaneous injections in the pons and corpus callosum of adult ALD mice induced consistently a neuronal expression of ALDP in distant specific areas, in agreement with diffusion of AAV vectors through neuron to neuron passage.

The presence of many ALDP positive neurons in the olfactory bulbs after AAV injection in the SVZ of newborn ALD mice indicated that AAV was able to transduce neural progenitors in the SVZ that differentiated in neurons and migrated to this brain area (Alvarez-Buylla et al., Prog. Brain Res., 2000, 127:1-11; Lim et al., Proc. Natl. Acad. Sci., U.S.A., 1997, 94:14832-14836). However, neurons expressing ALDP in thalamus, hippocampus, optic nuclei, pons, brain stem and even in the cerebellum, cervical spinal cord and opposite hemisphere, could not originate from transduced SVZ progenitors. Several of these distant brain areas expressing ALDP were identical in adult and newborn ALD mice, possibly because the AAV particles followed the same neuronal pathway from the SVZ and corpus callosum that are close to ventricles. Many ALDP positive neurons localized between remote areas and injections sites were lined up and connecting axons could be traced by immunostaining of peroxisomes with ALDP antibody. The spatial distribution of ALDP positive neurons far from injection sites suggests that they are synaptically connected, and that AAV particles are transported in axons from neurons transduced at the injection sites to remote neurons after transynaptic passage.

Numerous literature and patent references have been cited in the present application. All references cited are incorporated by reference herein in their entireties.

1. A method for delivering recombinant AAV virions to a subject, comprising:
selecting a first population and a second population of synaptically connected neurons, wherein a polypeptide of interest is to be expressed in said second population of neurons;
administering rAAV virions to said first subpopulation of neurons of said subject, wherein said rAAV virions comprise a nucleic acid sequence encoding said polypeptide of interest, and wherein said rAAV virions are capable of transducing synaptically connected neurons.

2. A method for delivering recombinant AAV virions to a subject, comprising:
identifying a subject suspected of suffering from, or susceptible to developing, a condition characterized by the degeneration of at least a first and a second specific neuronal population that are synaptically connected;
administering said rAAV virions intracerebrally such that rAAV virions are delivered to neurons of said subject, wherein said rAAV virions comprise a nucleic acid sequence encoding a therapeutic polypeptide.

3. The method of claim 2 wherein said rAAV virions are administered to said first subpopulation of neurons in said subject, wherein said rAAV virions are capable of being transported across at least one synapse between said first and said second populations of connected neurons.

4. The method of claim 1 wherein said first and second populations of neurons are separated by at least one synapse.

5. The method of claim 1 wherein said first and second populations of neurons are separated by at least two synapses.

6. The method of claim 1 wherein said first and second populations of neurons are separated by at least three synapses.

7. The method of claim 1 further comprising detecting the expression of said therapeutic polypeptide in a CNS cell of said subject.

8. The method of claim 1, further comprising detecting the transduction by said rAAV virions of a CNS cell of said subject.

9. The method of claim 1, wherein said rAAV virions transduce cells consisting essentially of neurons synaptically connected to one another.

10. The method of claim 1, wherein said polypeptide of interest is a therapeutic polypeptide and/or detectable polypeptide.

11. The method of claim 1 wherein said second population of neurons is a population of motor neurons.

12. The method of claim 1, wherein the administration comprises direct intracerebral administration.

13. The method of claim 1, wherein the administration comprises intrathecal administration.

14. The method of claim 1, wherein the administration comprises stereotactic microinjection.

15. The method of claim 1, wherein the subject is a human.

16. The method of claim 1, wherein the polypeptide is a non-secreted polypeptide.

17. The method of claim 1, wherein the polypeptide is a secreted polypeptide.

18. The method of claim 1, wherein the rAAV is a AAV-2, AAV-4 or AAV5 subtype.

19. The method of claim 1, wherein the nucleic acid sequence encodes a polypeptide capable of preventing or decreasing the rate of degeneration of a neuron.

20. A method for treating or preventing a neurodegenerative disease in a subject, said method comprising:
providing a preparation comprising recombinant adeno-associated virus (rAAV) virions, wherein said virions comprise a nucleic acid sequence that is expressible in transduced cells to provide a therapeutic effect in the subject; and
selecting a first population and a second population of synaptically connected neurons, wherein a therapeutic polypeptide is to be expressed in said second population of neurons;
delivering the preparation to said first population of neurons of the subject wherein said rAAV virions are capable of transducing synaptically connected neurons, and
wherein the nucleic acid sequence is expressed to provide a therapeutic effect in the subject suitable for treating said neurodegenerative disease.

21. The method of claim 20, wherein said neurodegenerative disease is Alzheimer's disease.

22. The method of claim 20, wherein said preparation is delivered to the corpus amygdaloideum of the subject.

23. The method of claim 20, wherein said preparation is delivered to the entorhinal cortex of the subject.

24. The method of claim 20, wherein the therapeutic polypeptide is a polypeptide capable of inhibiting or reducing the formation of Aβ production.

25. The method of claim 20, wherein the therapeutic polypeptide is a polypeptide capable of modifying APP processing.

26. The method of claim 20, wherein the therapeutic polypeptide is a polypeptide capable of stimulating α-secretase cleavage activity.

27. The method of claim 20, wherein the therapeutic polypeptide is a polypeptide capable of inhibiting the β-secretase pathway.

28. The method of claim 20, wherein the therapeutic polypeptide is a polypeptide capable of inhibiting the γ-secretase pathway.

29. The method of claim 20, wherein the therapeutic polypeptide is a polypeptide capable of inhibiting tau protein hyperphosphorylation.

30. The method of claim 20, wherein said rAAV virions comprise a nucleic acid sequence encoding an antisense nucleic acid or a catalytic RNA capable of reducing APP gene expression.

31. The method of claim 20, wherein said first and second populations of neurons are separated by at least one synapse.

32. The method claim 20, wherein said first and second populations of neurons are separated by at least two synapses.

33. The method of claim 20, wherein said first and second populations of neurons are separated by at least three synapses.

34. The method of claim 20, further comprising detecting the expression of said therapeutic polypeptide in a CNS cell of said subject.

35. The method of claim 20, further comprising detecting the transduction by said rAAV virions of a CNS cell of said subject.

36. The method of claim 20, wherein said rAAV virions transduce cells consisting essentially of neurons synaptically connected to one another.

37. The method of claim 20, wherein said therapeutic polypeptide is expressed in second population of neurons.

38. The method of claim 20, wherein the administration comprises direct intracerebral administration.

39. The method of claim 20, wherein the administration comprises intrathecal administration.

40. The method of claim 20, wherein the administration comprises stereotactic microinjection.

41. The method of claim 20, wherein the subject is a human.

42. The method of claim 20, wherein the polypeptide is a non-secreted polypeptide.

43. The method of claim 20, wherein the polypeptide is a secreted polypeptide.

44. The method of claim 20, wherein the rAAV is a AAV-2, AAV-4 or AAV5 subtype.

45. A method for treating or preventing a motor neuron disease in a subject, said method comprising:
providing a preparation comprising recombinant adeno-associated virus (rAAV) virions, wherein said virions comprise a nucleic acid sequence that is expressible in transduced cells to provide a therapeutic effect in the subject; and
selecting a first population and a second population of synaptically connected neurons, wherein a therapeutic polypeptide is to be expressed in said second population of neurons;
delivering the preparation to said first population of neurons of the subject wherein said rAAV virions are capable of transducing synaptically connected neurons, and
wherein the nucleic acid sequence is expressed to provide a therapeutic effect in the subject suitable for treating said a motor neuron disease.

46. The method of claim 45, wherein said motor neuron disease is amyotrophic lateral sclerosis (ALS).

47. The method of claim 45, wherein rAAV virions are delivered to the ruber nucleus.

48. The method of claim 45, wherein rAAV virions are delivered to the ventralis lateralis.

49. The method of claim 45, wherein rAAV virions are delivered to the anterior nuclei of the thalamus.

50. The method of claim 45, wherein said therapeutic polypeptide is superoxide dismutase 1 (SOD1).

51. The method of claim 45, wherein said therapeutic polypeptide is a polypeptide capable of inhibiting apoptotic cell death.

52. The method of claim 45, wherein said therapeutic polypeptide is a trophic factor.

53. The method of claim 45, wherein said motor neuron disease is SMA.

54. The method of claim 45, wherein said therapeutic polypeptide is SMN2.

55. The method of claim 45, wherein said therapeutic polypeptide is a trophic factor.

56. The method of claim 45, wherein said therapeutic polypeptide is a polypeptide capable of decreasing glutamate toxicity.

57. The method of claim 45, wherein said motor neuron disease is Kennedy's disease (bulbospinal atrophy).

58. The method of claim 45, wherein said therapeutic polypeptide is a chaperone polypeptide, or a polypeptide capable of increasing chaperone polypeptide expression.

59. The method of claim 45, wherein said therapeutic polypeptide is a trophic factor.

60. The method of claim 45, wherein said therapeutic polypeptide is a polypeptide capable of decreasing glutamate toxicity.

61. The method of claim 45, wherein said motor neuron disease is paraplegia.

62. The method of claim 45, wherein said first and second populations of neurons are separated by at least one synapse.

63. The method of claim 45, wherein said first and second populations of neurons are separated by at least two synapses.

64. The method of claim 45, wherein said first and second populations of neurons are separated by at least five synapses.

65. The method of claim 45, further comprising detecting the expression of said therapeutic polypeptide in a CNS cell of said subject.

66. The method of claim 45, further comprising detecting the transduction by said rAAV virions of a CNS cell of said subject.

67. The method of claim 45, wherein said rAAV virions transduce cells consisting essentially of neurons synaptically connected to one another.

68. The method of claim 45, wherein said first or second population of neurons comprises neurons of the CNS.

69. The method of claim 45, wherein said second population of neurons is a population of motor neurons.

70. The method of claim 45, wherein the administration comprises direct intracerebral administration.

71. The method of claim 45, wherein the administration comprises intrathecal administration.

72. The method of claim 45, wherein the administration comprises stereotactic microinjection.

73. The method of claim 45, wherein the subject is a human.

74. The method of claim 45, wherein the polypeptide is a non-secreted polypeptide.

75. The method of claim 45, wherein the polypeptide is a secreted polypeptide.

76. The method of claim 45, wherein the rAAV is a AAV-2, AAV-4 or AAV5 subtype.

77. The method of claim 45, further comprising administering to the subject at least one additional therapeutic compound.

78-101. (canceled)
